(19) United States
(12) Patent Application Publication
(10) Pub. No.: US 2025/0297943 A1
(43) Pub. Date: Sep. 25, 2025

(54) DEVICES, METHODS, AND SYSTEMS TO MEASURING AND RECORDING SPECTRUM OF A REACTANT ARRAY

(71) Applicant: SENSILL, INC., Concord, CA (US)

(72) Inventors: Richard Wyeth, Granite Bay, CA (US); Yan Zhou, Pleasanton, CA (US); William Shea, Martinez, CA (US)

(73) Assignee: SENSILL, INC., Concord, CA (US)

(21) Appl. No.: 19/229,664

(22) Filed: Jun. 5, 2025

Related U.S. Application Data
(63) Continuation of application No. PCT/US2023/083024, filed on Dec. 8, 2023.
(60) Provisional application No. 63/431,507, filed on Dec. 9, 2022.
(60) Provisional application No. 63/431,510, filed on Dec. 9, 2022.
(60) Provisional application No. 63/431,519, filed on Dec. 9, 2022.
(60) Provisional application No. 63/431,525, filed on Dec. 9, 2022.
(60) Provisional application No. 63/431,528, filed on Dec. 9, 2022.
(60) Provisional application No. 63/431,533, filed on Dec. 9, 2022.

(51) Int. Cl. G01N 21/25; G01N 21/31

(52) U.S. Cl. CPC G01N 21/255; G01N 21/31

(57) ABSTRACT

Devices, systems, and methods include a system comprising a light source directed at a surface, a spectrometer configured to measure, over time, levels of light collected from the surface, a controller in communication with the spectrometer. The controller may be configured to identify a component of fluid in contact with the surface based on the levels of the wavelengths of light collected from the surface.

### CROSS-REFERENCE TO RELATED APPLICATIONS

This application is a continuation of International Application No. PCT/US2023/083024, filed Dec. 8, 2023, which claims priority to: U.S. Provisional Patent Application Ser. No. 63/431,507, filed Dec. 9, 2022, the entirety of which is incorporated herein by reference; U.S. Provisional Patent Application Ser. No. 63/431,510, filed Dec. 9, 2022, the entirety of which is incorporated herein by reference; U.S. Provisional Patent Application Ser. No. 63/431,519, filed Dec. 9, 2022, the entirety of which is incorporated herein by reference; U.S. Provisional Patent Application Ser. No. 63/431,525, filed Dec. 9, 2022, the entirety of which are incorporated herein by reference; U.S. Provisional Patent Application Ser. No. 63/431,528, filed Dec. 9, 2022, the entirety of which are incorporated herein by reference; U.S. Provisional Patent Application Ser. No. 63/431,533, filed Dec. 9, 2022, the entirety of which are incorporated herein by reference.

### TECHNICAL FIELD

The present disclosure pertains to sensing and analysis tools, and the like. More particularly, the present disclosure pertains to devices and systems for sensing and analyzing chemical substances, and methods for manufacturing and using such devices.

### BACKGROUND

A wide variety of devices have been developed for collection, storing, sensing, and analysis of samples. These devices are manufactured by any one of a variety of different manufacturing methods and may be used according to any one of a variety of methods. Of the known medical devices and methods, each has certain advantages and disadvantages.

### BRIEF SUMMARY

This disclosure provides design, material, manufacturing method, and use alternatives for sensing and analysis devices. Although it is noted that collection, storing, sensing, and analysis approaches and systems are known, there exists a need for improvement on those approaches and systems.

An example system may include a spectrometer, a light source, and one or more fiber optic cables in communication with the spectrometer and configured to capture light reflected off a surface in response to illumination of the surface by the light source and deliver the captured light to the spectrometer, wherein the spectrometer may be configured to measure a photon count versus wavelength for each wavelength bin of an array of wavelength bins covering a light spectrum of interest from the light captured.

Alternatively or additionally to any of the embodiments in this section, the surface may be a surface of a color sensing array and the light captured includes light reflected from color bars of the color sensing array.

Alternatively or additionally to any of the embodiments in this section, the surface may be a surface of a color sensing array, the light captured may include light reflected from each color bar of a color sensing array, and the spectrometer may be configured to accurately measure and record reflectivity spectra of each of the color bars of the color sensing array independent of a spectral distribution of an intensity of the illumination from the light source.

Alternatively or additionally to any of the embodiments in this section, the array of wavelength bins may be a continuous linear array of wavelength bins.

In another example, a system may include a light source directed at a surface, a spectrometer configured to measure, over time, levels of wavelengths of light collected from the surface, and a controller in communication with the spectrometer, and wherein the controller may be configured to identify a component of fluid in contact with the surface based on the levels of the wavelengths of light collected from the surface.

Alternatively or additionally to any of the embodiments in this section, the light source may comprise a broadband white light emitting diode (LED).

Alternatively or additionally to any of the embodiments in this section, the light source may comprise a lens having a diameter and a focal length, wherein a ratio of the diameter to the focal length is one.

Alternatively or additionally to any of the embodiments in this section, the light source may comprise light having a wavelength spanning a range of 400 nanometers (nm) to 725 nm.

Alternatively or additionally to any of the embodiments in this section, the system may further include one or more optical fibers in communication with the spectrometer and configured to collect light from the light from the surface and direct the light collected to the spectrometer.

Alternatively or additionally to any of the embodiments in this section, the system may further include a light collection component configured to collect the light from the surface and an adjustable stage configured to be moved relative to the light collection component, and wherein the adjustable stage may be configured to support a component having the surface.

Alternatively or additionally to any of the embodiments in this section, the system may further include a motor in communication with the adjustable stage and configured to move the adjustable stage relative to the light collection component.

Alternatively or additionally to any of the embodiments in this section, the controller being configured to identify the component of fluid in contact with the surface based on the levels of the wavelengths of light collected from the surface may include the controller being configured to identify the component of fluid in contact with the surface based on one or more both of a timing of the levels of the wavelengths of light reflected off of the surface and an absolute change between a level of a wavelength of light collected from the surface at a time prior to an application of the fluid to the surface and at a predetermined time after initially applying the fluid to the surface.

In another example, a method may include adjusting a substrate along a light collection component in communication with a controller, wherein the substrate supports one or more reactants configured to change color in response to exposure to one or more fluids of interest, exposing the one or more reactants to a fluid, determining, over time with the controller, levels of wavelengths of light collected by the light collection component, and determining a component of the fluid based on the levels of the wavelengths of light collected.

Alternatively or additionally to any of the embodiments in this section, the method may further include applying light to the substrate, wherein the light includes light from a broadband white light emitting diode.

Alternatively or additionally to any of the embodiments in this section, applying light to the substrate may comprise applying light from a first angle and a first location with respect to the substrate and a second angle and a second location with respect to the substrate, where the second angle is the same as the first angle and the second location is different than the first location.

Alternatively or additionally to any of the embodiments in this section, adjusting the substrate along the light collection component may comprise passing the substrate along the light collection component a plurality of passes, wherein each pass of the plurality of passes includes passing a predetermined number of the one or more reactants on the substrate along the light collection component.

Alternatively or additionally to any of the embodiments in this section, the method may further include associating the levels of the wavelengths of light collected by the light collection component with the one or more reactants.

Alternatively or additionally to any of the embodiments in this section, determining a component of the fluid based on the levels of the wavelengths of light collected may include determining the component of the fluid based on one or both of a timing of the levels of the wavelengths of light collected as associated with the one or more reactants and an absolute change between levels of a wavelength of light collected as associated with the one or more reactants prior to the beginning of an application of the fluid to the one or more reactants and at a predetermined time after initially applying the fluid to the one or more reactants.

Alternatively or additionally to any of the embodiments in this section, determining, over time with the controller, the levels of the wavelengths of light collected by the light collection component may include determining levels of a spectra of the light collected spanning wavelengths in a range of 425 nm to 725 nm.

Alternatively or additionally to any of the embodiments in this section, determining the component of the fluid based on the levels of the wavelengths of light collected may comprise determining statistical data for levels of the wavelengths of light collected at a plurality of instances of time and comparing the statistical data determined to predetermined component statistical data.

In another example, a non-transitory computer readable medium having stored thereon a program code for use by a computing device, the program code causing the computing device to execute a method for determining a component of a fluid, the method comprising determining levels of wavelengths of light collected by a light collection component at one or more intervals, wherein the light collected during at least one interval is at least partially from one or more reactants, associating one or more of the levels of the wavelengths of light collected with one of the one or more reactants, comparing the levels of the wavelengths associated with the one or more reactants to predetermined sets of levels of the wavelengths for the one or more reactants, wherein each predetermined set is associated with a component of a fluid, and when the levels of the wavelengths associated with the one or more reactants matches a predetermined set of levels of the wavelengths for the one or more reactants, identifying the component of the fluid associated with the predetermined set of levels of the wavelengths for the one or more reactants.

Alternatively or additionally to any of the embodiments in this section, associating one or more of the levels of the wavelengths of light collected with one of the one or more reactants may comprise determining a time at which a minimum of the levels of the wavelengths of light collected occurred and associating a reactant of the one or more reactants with the levels of the wavelengths of light collected at the time.

Alternatively or additionally to any of the embodiments in this section, the determining the time at which the minimum of the levels of the wavelengths of light collected occurred and the associating a reactant of the one or more reactants with the levels of the wavelengths of light collected at the time may be repeated until all levels of the wavelengths of light collected at times associated with a minimum of levels are associated with a reactant of the one or more reactants or are discarded as being invalid minimums.

Alternatively or additionally to any of the embodiments in this section, the minimum of the levels of the wavelengths of light collected is a minimum of an average level of light over an entire spectrum of light collected.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic diagram of an illustrative sensing system;

FIG. 2 is a schematic diagram of an illustrative sensing system;

FIG. 3 is a schematic diagram of an illustrative computing system;

FIG. 4 is a schematic perspective view of an illustrative sensing system;

FIG. 5 is a schematic cross-section view of the illustrative sensing system of FIG. 4;

FIG. 6 is a schematic perspective view of an illustrative sensing system;

FIG. 7A is a schematic view along a first plane of an illustrative set of lenses for use in light collection;

FIG. 7B is a schematic view along a second plane of the illustrative set of lenses depicted in FIG. 7A, where the second plane is perpendicular to the first plane;

FIG. 8 is a schematic diagram of an illustrative technique for analyzing a sensed fluid;

FIGS. 9-11 are schematic graphs of sensed wavelengths of light from different reactants of an array of reactants exposed to a fluid during a fluid analysis test;

FIG. 12 is a schematic diagram of an illustrative technique for analyzing reactants;

FIG. 13 is a schematic graph of levels of wavelengths of light collected at a plurality of iterations or intervals during a fluid analysis test;

FIG. 14 is a schematic diagram of an illustrative technique for associating wavelength data with reactants of an array of reactants being sensed; and

FIG. 15 is a schematic graph of the average level of wavelengths of light collected at a plurality of intervals during a fluid analysis test.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

The term “fluid” is inclusive of both liquids and gases.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an configuration”, “some configurations”, “other configurations”, etc., indicate that the configuration described may include one or more particular features, structures, and/or characteristics. However, such recitations do not necessarily mean that all embodiments include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Fluids with concentrations of volatile compounds (e.g., volatile organic compounds (VOCs)) and/or gasses, which may or may not be hazardous, may be sensed, analyzed, and/or monitored. Sensing, analyzing, and/or monitoring of fluids with analytes (e.g., non-volatile and/or volatile compounds, gases, liquids, and/or other fluids) may utilize absorption measurements of reactants (e.g., an analyte sensitive material) exposed to such fluids for any purpose including, but not limited to, diagnostic hazard warning, manufacturing process or quality control, record keeping archival purposes, product development, product-consumer matching, etc.

In some cases, VOCs and/or gasses may be present in ambient fluid (e.g., ambient air, etc.) and sensed, analyzed, and/or monitored using reactants for real-time alarms, to treat subjects, or to collect and/or archive data for health records, regulatory compliance records, etc. Further, VOCs and/or gasses exhaled or emitted, excreted, emanated, released, and/or secreted from a subject (e.g., humans, animals other than humans, food, produce, meat, pathogens, bacteria (e.g., good and/or bad bacteria), plants, wounds, ulcers, surgical sites, skin of a subject, mouth of a subject, nasal passages of a subject, sinuses of a subject, rectum area of a subject, vaginal area of a subject, genitals area of a subject, car canals of a subject, pores of a subject, etc.) may be sensed, analyzed, and/or monitored to assess hazardous, dangerous, or illegal substances in or at the subject or target site, a lung condition of lungs of a subject, a condition of a blood disease, a condition of infections, conditions related to diseases or biological conditions, conditions related to general health, conditions related to food flavors, conditions related to perfumes or smells, and/or other suitable conditions.

The systems discussed herein for sensing, analyzing, and/or monitoring fluids (e.g., for analytes of interest) may be configured to accurately detect and record a colorimetric sensor array (CSA) spectral response to exposure to the fluids. The systems may utilize techniques for non-invasively detecting one or more analytes of interest (e.g., one or more pathogens responsible for specific human skin infections including, but not limited to, skin infections, urinary tract infections (UTIs), vaginitis, wound infections, ulcers, etc., and/or other suitable analytes) from a fluid using a CSA to allow for early detection of and early implementation of protocols to address one or more conditions associated with any sensed analytes of interest. In one example, enhanced classification of one or more analytes using the systems described herein may enable detection and identification of responsible pathogens at the very beginning stages of a dangerous skin infection, which may result in a high level of protection and probability of a favorable outcome for subjects.

The systems for sensing, analyzing, and/or monitoring analytes of fluids may use optics to capture photons diffused, reflected, scattered, transmitted, or reemitted from individual reactants (e.g., color areas, color imprints, color bars, color dots, etc.) applied to a substrate or membrane of a CSA and deliver the photons via a fiber optic cable or free space optics to a high-resolution spectrometer having a photodetector (e.g., a silicon photodetector and/or other suitable photodetector) for measurement of photon count versus wavelength for each of a continuous array (e.g., linear, multidimensional and/or other suitable shape or configuration of an array) of wavelength bins covering the light spectrum of interest (e.g., visible light spectrum, ultraviolet (UV) light spectrum, infrared (IR) light spectrum, etc.) Appropriate calibration techniques and an algebraic signal processing algorithm may be applied to the measurements to calculate a light collection measurement (e.g., reflectivity, photon count, etc.) at individual consecutive wavelengths across the electromagnetic wave spectrum. This technique may be applicable for wavelengths extending from the ultra-violet, through the visible, and into the mid-infrared portion of the spectrum. In some cases, a motion stage (e.g., an adjustable stage) may be employed to facilitate collecting multiple spectra at discrete locations over a full reactant array of the CSA.

The systems for sensing, analyzing, and/or monitoring components of a fluid (e.g., analytes of interest, etc.) may capture and process data iteratively or continuously on-the-fly as the entire reactant array or an entirety of a portion of the reactant array of the CSA is viewed for processing. The captured or obtained data (e.g., spectral data) may then be processed to accurately associate spectra data with each reactant in the CSA. During a single fluid analysis test, the reactant array or a portion of the reactant array may be viewed for processing multiple times. By performing repetitive measurements over time, the changes to the reflective spectra of some or all reactants of a reactant array (e.g., reactant array of a CSA) may be recorded during exposure of the reactant array to a fluid and used to identify components of the fluid (e.g., analytes of interest).

The systems for sensing, analyzing, and/or monitoring fluids may facilitate accurately recording the reflectivity spectra for each individual reactant of a reactant array in a manner that is independent of the spectral distribution of a light source intensity illuminating the reactants of the reactant array or spectral variation of the response of the photon intensity measurement device (e.g., of the spectrometer). For example, high wavelength resolution and continuous spectral response of a spectrometer of the system may produce an accurate description of the wavelength content of the light from each reactant of the reactant array that is independent of variables associated with the system and/or environment. Existing devices use electronic imaging chips that collect values for only a small number of relatively narrow spectral ranges such as specific red, blue, and green filtered portions of the visible spectrum. In contrast, the systems for sensing, analyzing, and/or monitoring fluids utilizing a spectrometer as discussed herein may record over 700 spectral content-versus-wavelength data point values over the continuous visible light spectrum or beyond (e.g., over the infrared (IR) spectrum, ultraviolet (UV) spectrum etc.), which may facilitate detecting small changes to light quantity (e.g., photon count) versus wavelength data points (e.g., measurements) of one or more reactants in response to exposure of the reactants to a fluid having a component of interest (e.g., one or more analytes of interest). By improving the quality and quantity of the data describing physical properties that determine the original color and the color change of the individual reactants in a reactant array of a CSA, the classification of spectral data before, during, and after exposure of the CSA to fluids with various analytes of interest by the systems, described herein, for sensing, analyzing, and/or monitoring analytes is enhanced over existing systems.

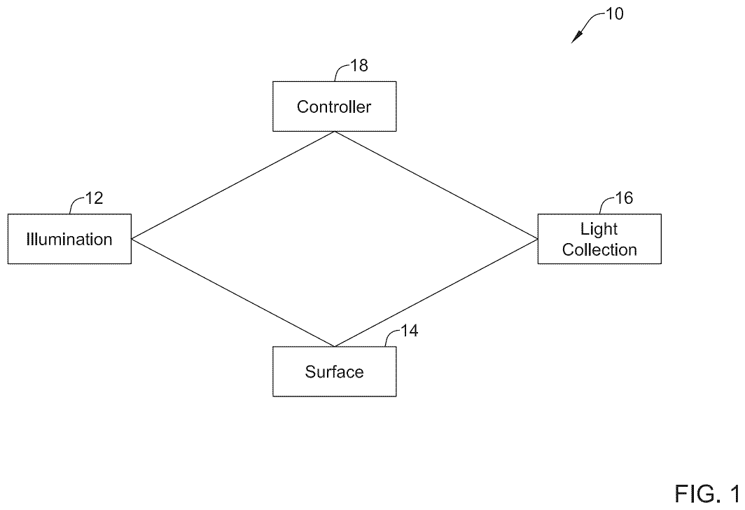

Turning to the Figures, FIG. 1 schematically depicts an illustrative configuration of a fluid analysis system 10 for determining a component of a fluid. In some examples, the fluid analysis system 10 may include, among other components, an illumination component 12 configured to illuminate one or more reactants (e.g., an analyte sensitive material) of a reactant array on or otherwise supported by a surface 14, a light collection component 16 configured to receive or collect light from the one or more reactants, and a controller 18 configured to be in communication with the illumination component 12 and/or the light collection component 16. In some examples, the illumination component 12 and/or the light collection component 16 may form or be part of an optical system of the fluid analysis system 10. The controller 18 may be configured to analyze or facilitate analyzing data related to light collected at the light collection component 16.

The one or more reactants of the reactant array on or supported by the surface 14 may be exposed to fluid. In some examples, the one or more reactants may be exposed to fluid in any suitable manner including, but not limited to, by pumping fluid to or along the one or more reactants during a fluid test using the fluid analysis system 10, exposing the one or more reactants to the fluid prior to being positioned in the fluid analysis system 10, positioning the one or more reactants proximate an area of interest (e.g., a wound, etc.) prior to being positioned in the fluid analysis system, and/or the one or more reactants may be exposed to fluid in one or more other suitable manners. Once the one or more reactants have been exposed to fluid for analysis of the fluid and light has been collected from the one or more reactants during a fluid analysis test, the controller 18 may analyze light collection data to identifying one or more components (e.g., analytes of interest) of the fluid to which the one or more reactants were exposed.

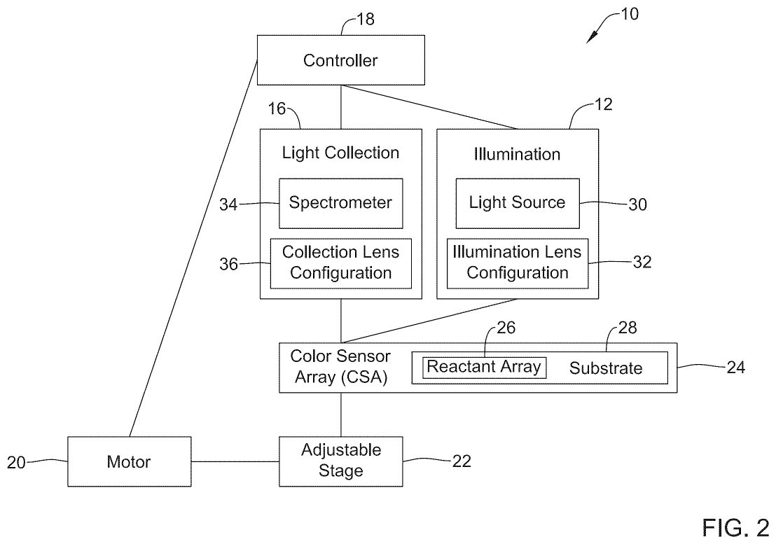

FIG. 2 schematically depicts a diagram of an illustrative configuration of the fluid analysis system 10 including the illumination component 12, the light collection component 16, and the controller 18. In some examples, the fluid analysis system 10 may additionally include a motor 20 in communication with the controller 18 and an adjustable stage 22 including or coupled with a detecting component (e.g., a colorimetric sensor array (CSA) 24). The CSA 24, when included as the detecting component, may include a reactant array 26 having the one or more reactants and a substrate 28 supporting the reactant array 26. In some cases, the substrate 28 may be or may include the surface 14 depicted in FIG. 1, but other configurations are contemplated. Optionally, the fluid analysis system 10 may include a housing configured to house one or more of the illumination component 12, the surface 14, the light collection component 16, the controller 18, the motor 20, the adjustable stage 22, the CSA 24, and/or other suitable components of the fluid analysis system 10.

The CSA 24 may be configured in the fluid analysis system 10 to be adjusted relative to the illumination component 12 and/or the light collection component 16 to facilitate collecting light from all of or a desired amount of the reactants of the reactant array 26. In one example, the CSA 24 may be adjusted relative to the illumination component 12 and/or the light collection component 16 in response to actuation of the motor 20 such that different reactants are selectively positioned at a target area of the illumination component 12 and/or the light collection component 16. In one example, the motor 20 may be in communication with the adjustable stage 22 such that actuation of the motor 20 may cause the adjustable stage 22 to adjust and move (e.g., translate, rotate, etc.) the CSA 24 relative to the illumination component 12 and/or the light collection component 16 (e.g., relative to the target area of the illumination component 12 and/or the light collection component 16), where the illumination component 12 and the light collection component 16 may be fixed relative to one another and other components of the fluid analysis system 10. Alternatively or additionally, one or both of the illumination component 12 and the light collection component 16 may be adjusted relative to the CSA 24 in response to actuation of the motor 20.

The motor 20 may be any suitable type of device configured to couple with and adjust a position of the adjustable stage 22 and/or the CSA 24 relative to the illumination component 12 and/or the light collection component 16. For example, the motor 20 may be a stepper motor, a continuous drive motor, a direct current (DC) motor, a servo motor, a manually operated handwheel, and/or other suitable device or system configured to produce motion. In some cases, the motor 20 may include a drive shaft configured to drive a driven component (e.g., the adjustable stage 22 and/or the CSA 24 or other suitable driven component coupled with the adjustable stage 22 and/or the CSA 24).

The motor 20 may be coupled with the adjustable stage 22 in any suitable manner to facilitate a desired adjustment (e.g., linear adjustment, rotational adjustment, linear and rotational adjustment, and/or other suitable adjustment) of the adjustable stage 22 in response to actuation of the motor 20. When the adjustable stage 22 and/or the CSA 24 are to be adjusted in a linear manner, the coupling between the motor 20 and the adjustable stage 22 may facilitate transferring the rotational motion of the motor 20 into linear motion of the adjustable stage 22. When the adjustable stage 22 and/or the CSA 24 are to be adjusted in a rotational manner, the coupling between the motor 20 and the adjustable stage may facilitate transferring rotational motion of the motor 20 into rotational motion of the adjustable stage 22. When the adjustable stage 22 and/or the CSA 24 are to be adjusted in a linear manner and a rotational manner, the coupling(s) between the motor 20 and the adjustable stage 22 may facilitate transferring rotational motion of the motor 20 into linear motion of the adjustable stage 22 and rotational motion of the adjustable stage 22.

The motor 20 and the coupling with the adjustable stage 22 may be configured to adjust a position of the adjustable stage 22 and/or the CSA 24 at any suitable speed or rate. In some examples, the motor 20 may be configured to adjust the adjustable stage 22 and/or the CSA 24 at a speed or rate in a range of less than 1 millimeter (mm)/second(s), in a range of about 1 mm/s to about 20 mm/s, in a range of 20 mm/s or greater, but other suitable ranges are contemplated. In some configurations, the motor 20 may be configured to continuously adjust the adjustable stage 22 and/or CSA 24 at a constant speed or rate and/or change a speed or rate during fluid test. In one example configuration, the motor 20 may be configured to adjust a position of the adjustable stage 22 and/or the CSA 24 at a constant speed or rate of 5 mm/s during a fluid test.

The adjustable stage 22 may be any suitable component configured to support the CSA 24 and/or reactant array 26 (e.g., where the CSA 24 may be a component having the surface 14). In some examples, the adjustable stage 22 may be or may include a platform coupled with the motor 20 (e.g., coupled directly or indirectly via a drive shaft of or extending from the motor 20) and configured to support one or more CSAs 24 including the reactant array 26 as the adjustable stage 22 is moved relative to the light collection component 16. Additionally or alternatively, the adjustable stage 22 may be or may include an arm coupled with the CSA 24 and the motor 20 to transfer motion of the motor 20 to the CSA 24. Further, in some examples, the adjustable stage 22 may be or may include the CSA 24. For example, the adjustable stage 22 may be or may include the substrate 28 on which the reactant array 26 is located and as a result, may include the CSA 24.

The substrate 28 of the CSA 24 may have any suitable configuration for supporting and/or receiving the reactant array 26 for exposure to a fluid (e.g., a fluid of interest) and/or for analysis of the reactant array using the optical system of the system 10. For example, the substrate 28 may be sized to contain all of or a portion of the reactant array 26. In some examples, multiple substrates 28 may be utilized to contain all of or a portion of the reactant array 26. The substrate 28 may take on, or may have a surface (e.g., the surface 14) that may be, any suitable shape including, but not limited to, an elongated shape, a rectangular shape, a square shape, a rounded shape, a circular shape, a cylindrical shape, a disc shape, and/or other suitable shape. The substrate 28 may be or include a surface of a container or cartridge or a component configured to be within a container or cartridge

The substrate 28 may include and/or may be formed from any suitable material. Example suitable materials used for the substrate 28 of the CSA 24 include, but are not limited to, polymers, plastic, rubber, glass, paper, filter material, filter paper, fabric, metal, aluminum, polypropylene, polytetrafluorethylenes, porous membranes, chromatography plates, other suitable materials, and/or combinations thereof. Further, the material utilized for the substrate 28 may be a solid material, a woven material, a hydrophobic material, a gas permeable material, a gas impermeable material, other suitable materials, and/or combinations thereof.

In one example configuration of the substrate 28, the substrate 28 may be a porous white plastic membrane that has a high diffuse reflectivity over an entire visible spectrum, at least a portion of the ultraviolet (UV) spectrum, and/or at least a portion of the infrared (IR) spectrum. When the substrate 28 is formed from a white plastic membrane that has a high diffuse reflectivity over at least an entire visible spectrum, the light collection component 16 of the fluid analysis system 10 may be configured to collect a 100% white spectrum from the substrate 28, which may be used for fluid analysis purposes as discussed in greater detail herein.

In another example configuration of the substrate 28, the substrate 28 may be formed from a woven polypropylene material, which may result in a gas permeable, hydrophobic substrate 28. Although other pore sizes are contemplated, in the example configuration, the woven substrate may have an average pore size of or about 0.2 microns and a diameter of about 25 millimeters (mm). Additionally or alternatively, an example configuration of the substrate 28 may be formed from a hydrophobic, gas permeable material. Such a configured substrate 28 may be comprised of one or more gas permeable materials that provide a desired set of structural properties and gas permeability.

To increase fluid component detection rates by the reactants of the reactant array 26, the substrate 28 on which the reactant array 26 is applied and/or the reactants of the reactant array 26 may be textured (e.g., with grooves or surface topographical undulations, woven patterns, etc.) so as to increase an effective surface area of the reactants. Such texturing may be applied to substrate 28 using any suitable technique including, but not limited to, via etching, thermoforming, pressure forming, molding, machining, weaving, three-dimensional printing, and/or other suitable techniques.

In some instances, the substrate 26 may be omitted or incorporated into the reactant array 26. In such instances, the reactants of the reactant array 26 may form a structure on their own that is configured to react to fluid exposure and that can be analyzed with the fluid analysis system 10.

The reactants of the reactant array 26 may be formed from any suitable material. In some cases, the material of the reactants may be an optically responsive chemical material (e.g., a chemoresponsive material) that changes color in response to detecting one or more analytes (e.g., non-volatile and/or volatile compounds, gases, liquids, and/or other fluids) in a fluid to which the reactants are exposed. Example suitable materials for reactants include dyes from, but not limited to, the following classes: Lewis acid/base dyes (e.g., metal ion containing dyes), Brensted acidic or basic dyes (e.g., pH indicators), dyes with large permanent dipoles (e.g., solvatochromic dyes), redox responsive dyes (e.g., metal nanoparticle precursors), and/or other suitable classes of dyes. One example material for the reactants may be a silver nanoparticle material. Other suitable materials for the reactants are contemplated, including reactant material that is not a printed dye.

In some examples, the material of the reactants may include an analyte sensitive material that is reversible or semi-reversible. Reversible or semi-reversible analyte sensitive material may be utilized for reactants configured for repeat monitoring, such as for continuous or periodic sensing of target locations to detect analytes from the target locations. Although other configurations of reactant arrays 26 are contemplated, example reactant arrays 26 including analyte sensitive material that is reversible or semi-reversible are discussed in U.S. Pat. No. 6,368,558 filed on Mar. 21, 2000, and titled COLORIMETRIC ARTIFICIAL NOSE HAVING AN ARRAY OF DYES AND METHOD FOR ARTIFICIAL OLFACTION; U.S. Pat. No. 6,495,102 filed on Nov. 11, 2000, and titled COLORIMETRIC ARTIFICIAL NOSE HAVING AN ARRAY OF DYES AND METHOD FOR ARTIFICIAL OLFACTION; U.S. Pat. No. 7,261,857 filed on Oct. 24, 2002, and titled COLORIMETRIC ARTIFICIAL NOSE HAVING AN ARRAY OF DYES AND METHOD FOR ARTIFICIAL OLFACTION; U.S. Pat. No. 8,852,504 filed on Oct. 11, 2007, and titled APPARATUS AND METHOD FOR DETECTING AND IDENTIFYING MICROORGANISMS, all of which are hereby incorporated by reference in their entirety and for all purposes.

In some examples, the material of the reactants may include an analyte sensitive material that is irreversible. Irreversible analyte sensitive material may be utilized for reactants configured for single use monitoring or single use monitoring per analyte material of a fluid when the reactant array 26 is configured to monitor for a plurality of different analytes, but this is not required. Although other configurations of reactant arrays 26 are contemplated, example reactant arrays 26 including analyte sensing material that is irreversible are discussed in U.S. Pat. No. 9,880,137 filed on Sep. 2, 2009, and titled COLORIMETRIC SENSOR ARRAYS BASED ON NANOPOROUS PIGMENTS; U.S. Pat. No. 10,539,508 filed on Jun. 9, 2015, and titled PORTABLE DEVICE FOR COLORIMETRIC OR FLUOROMETRIC ANALYSIS AND METHOD OF CONDUCTING COLORIMETRIC OR FLUOROMETRIC ANALYSIS; Li, Zheng, et al., “Ultrasensitive Monitoring of Museum Airborne Pollutants Using a Silver Nanoparticle Sensor Array”, ACS sensors 5.9 (2020): 2783-2791; Li, Zheng, and Kenneth S. Suslick, “Chemically Induced Sintering of Nanoparticles”, Angewandte Chemie 131.40 (2019): 14331-14334; LaGasse, Maria K., et al., “Colorimetric sensor arrays: Development and application to art conservation”, Journal of the American Institute for Conservation 57.3 (2018): 127-140, all of which are hereby incorporated by reference in their entirety and for all purposes.

The reactants of the reactant array 26 may be applied to the substrate 28 in any suitable manner. In one example, the reactants may be applied to the substrate 28 by printing the reactants (e.g., the material of the reactants) on the substrate 28. When printed, any suitable printing techniques may be utilized including, but not limited to, pin transfer, inkjet, silkscreen, and/or other suitable application techniques.

The reactants may be applied to the substrate 28 randomly and/or to form one or more patterns. Example configurations of the reactants of the reactant array 26 applied to the substrate 28 include, but are not limited to, grid patterns of rows and columns, concentric or spiral rings, color matching of a color of printed dye material with a color of a substrate material prior to interactions with analyte, patterns that result in identifiable shapes when the analyte sensitive material reacts to a particular analyte, other suitable configurations, and/or combinations thereof.

The fluid analysis system 10 may include an optics system configured to facilitate collecting photons to calculate a light collection measurement (e.g., reflectivity, photon count, etc.) of individual reactants of the reactant array 26. As discussed above, the optics system may include the illumination component 12 and/or the light collection component 16, among other suitable components.

In some examples, the optics system or a portion thereof may be configured to be stationary relative to the adjustable stage 22 and/or the CSA 24. Alternatively or additionally, the optics system or a portion thereof may be configured to move or otherwise adjust relative to the adjustable stage 22 and/or the CSA 24. In some configurations of the fluid analysis system, the adjustable stage 22 may be omitted and the CSA 24 may be stationary as the optics system or a portion thereof is adjusted. Alternatively, the optics system or a portion thereof and the CSA 24 may be stationary (e.g., fixed) relative to one another.

When included in the fluid analysis system 10, the illumination component 12 may include one or more light sources 30, an illumination lens system 32 (e.g., an illumination lens subsystem), and/or other suitable components. The illumination component 12 may be configured to provide sufficient photons with a uniform spatial and spectral distribution spanning a wavelength range of interest for the CSA 24 to the reactants of the reactant array 26.

To maximize a signal to noise ratio for collecting light from the reactants of the reactant array 26 while minimizing consumed electrical power, an efficiency of electron to photon conversion of the one or more light sources 30 may be of interest. Also, efficiency in maximizing a ratio of collected photons to illumination photons may be considered. To facilitate maximizing the ratio of collected photons to illumination photons, the distribution of photons over the wavelength range of interest from the light sources 30 may be uniform. As such, utilizing an energy efficient light source that provides a uniform distribution of photons over the wavelength range of interest facilitates obtaining or calculating an accurate low noise light collection measurement (e.g., reflectivity, photon count, etc.) in every wavelength bin of the light collection component 16.

The one or more light sources 30 may be configured to provide any suitable wavelengths of light to one or more reactants. In some examples, the one or more light sources 30 may provide uniform spatial and spectral distributions of wavelengths of light spanning one or more ranges of about 300 nanometers (nm) to about 1000 nm, a range of about 360 nm to about 900 nm, a range of about 300 nm to about 600 nm, a range of about 350 nm to about 500 nm, a range of about 400 nm to about 725 nm, a range of about 425 nm to about 725 nm, a range of 700 nm to about 1000 nm, a range of about 800 nm to about 1000 nm, and/or other suitable ranges of wavelengths of light. In one example, one or more light sources 30 may provide wavelengths of light spanning a range of about 400 nm to about 725 nm.

The optics system may be configured to provide illumination light in two or more different discrete ranges of wavelengths of light. For example, the one or more light sources 30 may provide light in a first range of wavelengths of light (e.g., about 300 nm to about 600 nm) and in a second range of wavelengths of light (e.g., about 800 nm to about 1000 nm). The optics system may provide illumination in such two discrete ranges of wavelengths of light by utilizing two or more light sources 30, through the use of filters, and/or in one or more other suitable manners. Having the ability to provide light in two or more discrete wavelength ranges may facilitate using the fluid analysis system 10 for different applications that may require use of different wavelength ranges for optimal performance (e.g., optical detection of fluid components and/or other analytes).

In some configurations, the one or more light sources 30 may be configured to provide at least a uniform spatial and spectral distribution of broadband white light (e.g., continuous broadband white light) to one or more reactants of the reactant array 26. In one example, the light source 30 providing the uniform spatial and spectral distribution of broadband white light may provide light wavelengths spanning a range of about 360 nm to about 900 nm. In another example, the light source 30 providing the uniform spatial and spectral distribution of broadband white light may provide light wavelengths spanning a range of about 400 nm to about 725 nm. Such configured light sources 30 may have a desired (e.g., high) color rendering index (CRI), with a uniform distribution of photon wavelengths through the entire visible spectrum.

The one or more light sources 30 may be any suitable type of light source. For example, the light source 30 may be a light emitting diode (LED), an indium based blue LED with multiple phosphors added to a doping to create a combined LED and electro-luminescent semiconductor junction light emitting source, a black body radiation source, a tungsten lamp, a halogen lamp, and/or other suitable type of light source 30. In one example, the light source 30 may be a true color white LED configured to provide light wavelengths in a range of about 400 nm to about 725 nm, but other suitable configurations are contemplated. Utilizing a white LED rather than a black body radiation source (e.g., tungsten lamps, halogen lamps, etc.) may reduce inefficiencies of electron to photon conversion and allow the fluid analysis system 10 to use less power (e.g., have a higher electron to photon conversion ratio) than when other types of light sources 30 (e.g., tungsten lamps, halogen lamps, etc.) are used.

The light sources 30 may be provided at any suitable angle and at any suitable location relative to the CSA 24 (e.g., the reactant array 26 of the CSA 24) and/or the light collection component 16. For example, the light sources 30 may be provided at angles in a range of about 15 degrees to about 75 degrees relative to the CSA 24, at angles in a range of about 30 degrees to about 60 degrees relative to the CSA 24, at angles in a range of about 40 degrees and 50 degrees relative to the CSA 24, and/or at one or more other suitable angles. In one example, the light sources 30 may be angled at 45 degrees relative to the CSA 24, but other suitable configurations are contemplated. Providing light sources 30 that project light onto the reactants of the reactant array 26 from an acute angle and from a location spaced laterally from a target area (e.g., a lighted area) on the CSA 24 may facilitate providing dual overlapping ellipsoids that effectively form the target area (e.g., form a target area sized to cover one or more reactants or portions of the one or more reactants) to be analyzed while minimizing collection of spectral or specular reflection light and allowing for maximum diffuse light collection.

The one or more light sources 30 may be configured in any suitable manner relative to the CSA 24. In some examples and as discussed, the one or more light sources 30 may be configured relative to the CSA 24 such that illumination may be projected on the CSA 24 in a manner that prevents or mitigates spectral or specular reflections being captured by the light collection component 16 and maximizes capturing diffuse light from the CSA 24 (e.g., reflections, etc. from the reactants of the reactant array 26). In one example, the one or more light sources 30 may include a first light source 30 and a second light source 30, where the first and second light sources 30 may be identical or different from one another and may be configured to illuminate a same target area on the CSA 24 (e.g., where the target area may be formed to cover one or more reactants or portions of one or more reactants of the reactant array 26). In some examples, the first and second light sources 30 and/or other light sources 30 may be positioned at a same angle relative to the CSA 24 and at different locations relative to the CSA 24, but other suitable configurations are contemplated.

In one example configuration of light sources 30 including the first and the second light sources 30, the first light source 30 may be at a first location and a first angle relative to the CSA 24 and the second light source 30 may be at a second angle and a second location relative to the CSA 24. The first angle and the second angle may be a same angle or a different angle. In one example, the first angle and the second angle may be a same angle and may be about 45 degrees relative to the CSA 24 (e.g., relative to the reactant array 26 or surface supporting the reactant array 26). The first location and the second location may be different locations and in one example, the first location and the second location may oppose one another such that light from a same angle, but opposite directions, is applied to the CSA 24 to form a target area on the CSA 24. Other suitable configurations are contemplated.

The illumination lens system 32, when included, may be configured to deliver light from the light source 30 to, and focus the light on, the desired target area on the CSA 24. In some examples, the target area on the CSA 24 may be one or more reactants of the reactant array 26, but other suitable target areas are contemplated. The illumination lens system 32 may include any suitable components including, but not limited to, one or more lenses, one or more fiber optics, and/or one or more other suitable components.

When one or more fiber optics (e.g., one or more optical fibers) are included in the illumination lens system 32, the fiber optics may be configured (e.g., tuned and positioned) to deliver light to or focus light on one or more reactants of the reactant array 26 from the light source(s) 30. The fiber optics may be single mode and/or multimode fiber optics, as desired.

The one or more lenses, when included in the illumination lens system 32, may be configured (e.g., tuned and positioned) to deliver light to or focus light on the target area (e.g., one or more reactants of the reactant array 26) from the light source(s) 30. The one or more lenses may include a configuration with a single lens or a configuration with two or more lenses that may be similar or different than one another. In some examples, the illumination lens configuration may include a first lens and a second lens that may operate together to deliver light to or focus light on the target area. In one example, the first lens may be located between the light source 30 and the second lens and may be a convex lens (e.g., a spherical lens) configured to collimate light from the light source 30 and the second lens may be located between the first lens and the target area on the CSA 24 and may be a convex lens or other suitable lens configured to focus light from the light source 30 onto the target area of the CSA 24.

In some examples, the one or more lenses of the illumination lens system 32 may have a diameter and a focal length, where a ratio of the diameter to the focal length (e.g., a lens F number) may be in a range of about 0.5 to about 2.0, in a range of about 0.75 to about 1.5, in a range of about 0.8 to about 1.2, in a range of about 0.9 to about 1.1, and/or within one or more other suitable range. In one example, the one or more lenses of the illumination lens system 32 may have a diameter to focal length of about 1.0. Further, although not required, the one or more lenses may include a short focal length convex lens that may be configured to match a spot size on the CSA 24 from a light source 30 with a size of a target area (e.g., one or more reactants) on the CSA 24.

The light collection component 16 (e.g., diffuse reflection capture optics, etc.) may be configured to collect and measure levels of wavelengths of light collected from the surface 14 (e.g., measure photons by wavelengths of light from individual reactants of the reactant array 26) and may include one or more light collectors, a collection lens system 36 (e.g., a collection lens subsystem), and/or other suitable components. The light collection component 16 may be configured to be focused on the target area (e.g., a reactant of the reactant array 26) to avoid or mitigate collecting light from spaces (e.g., white spaces) between reactants and/or from more than one reactant. Further, focusing the light collection component on a single reactant may facilitate obtaining light from an entirety of or at least a majority of the single reactant, which may minimize the likelihood of obtaining skewed light measurements from the reactant due to printing defects, granularity in the material used for the reactants, defects in the substrate 28, and/or due to other irregularities. In some examples, at least a portion of the light collection component 16 (e.g., a portion of the light collection component 16 proximate the CSA 24) may be oriented perpendicular to or substantially perpendicular to substrate 28 and/or a surface of the CSA 24 to minimize collecting or receiving light from spectral or specular reflections and maximize light collection from the target area.

The collection lens system 36, when included, may be configured to receive or collect light from the target area of the CSA 24 and focus an aperture on the target area (e.g., the focus of the aperture may be slightly smaller than the illumination spot from the illumination component 12). The collection lens system 36 may include any suitable components including, but not limited to, one or more lenses, one or more fiber optics, and/or one or more other suitable components.

When one or more fiber optics (e.g., one or more optical fibers) are included in the collection lens system 36, the fiber optics may be configured (e.g., tuned and positioned) to receive light from or focus light from one or more reactants of the reactant array 26. The one or more fiber optics may be or may include single mode and/or multimode fiber optics, as desired. The one or more fiber optics may have a first end configured to receive or collect light from the target area and a second end in optical communication with a light collector.

The one or more lenses, when included in the collection lens system 36, may be configured (e.g., tuned and positioned) to receive or collect light from the target area (e.g., one or more reactants of the reactant array 26) and direct the light to an image sensor or wave guide in optical communication with a sensor or other component of the light collector. The one or more lenses may include a configuration with a single lens or a configuration with two or more lenses that may be similar or different than one another. In some examples, the collection lens system 36 may include a first lens and a second lens that may operate together to obtain light from the target area (e.g., light from substantially only the target area) and deliver light to or focus light on a sensor of the light collector.

In one example configuration of the collection lens system 36 with two lenses and a fiber optic wave guide in communication with the light collector, the first lens may be located between the light collector or a wave guide (e.g., a fiber optic) in optical communication with a sensor of the light collector and may be a convex lens (e.g., an aspheric lens) configured to focus light from the target area of the CSA 24 (e.g., via the second lens) on the sensor and/or an inner core of the wave guide. The second lens may be located between the first lens and the target area on the CSA 24 and may be a cylinder lens or other suitable lens configured to collect light from an entirety of or substantially an entirety of the target area on the CSA 24 (e.g., a reactant of the reactant array 26).

A focal length of the combination of first lens and the second lens, a distance from the second lens to the target area, and a distance to an inner core of the fiber optic wave guide may be chosen or selected to give precise magnification and dimensions required for an acceptance aperture configured to optimize overall illuminator to light collector photon utilization efficiency, minimize electrical power required to sense light from the CSA 24, and ensure the light collector integration time may be minimized. In turn, this may allow a sample rate of the light collector to be increased, which may reduce an amount of time needed to capture all of the individual spectra required to fully characterize the response of every reactant or at least a desired subset of reactants in the reactant array 26 for use in analyzing a fluid sensed by the reactant array 26.

The one or more light collectors may be any suitable type of light collector. Example suitable types of collectors may include, but are not limited to, an image sensor, a spectrometer 34, a charge-coupled device (CCD) image sensor, complementary metal-oxide semiconductor (CMOS) image sensor, color contact image sensor (CCIS), other suitable light collectors, and/or combinations of light collectors. In one example, the light collection component 16 may include a spectrometer 34 configured to measure photons collected from (e.g., reflected, transmitted, and/or otherwise received from) the target area. Utilizing the spectrometer 34 may facilitate sensing wavelengths of light with high resolution in the nanometer range and may provide a continuous set of data over the wavelength range, which allows for a sensitive analysis of the data to identify components of a fluid to which the reactant array 26 was exposed relative to when other light collectors are used. In some examples, the spectrometer 34 may be tuned or otherwise configured to have one nm or greater bin widths, which may result in the measurements or data from the spectrometer 34 having a desirable resolution that contributes to the detection sensitivity of the fluid analysis system 10.

Any suitable type of spectrometer 34 configured to measure (e.g., measure over time) levels of wavelengths of light collected from the surface 14 (e.g., from the CSA 24) may be utilized as the light collector. In some examples, the spectrometer 34 may have a compact folded optical system with a diffraction grating and a linear imager (e.g., a linear array photo detector, a CCD linear imager, and/or other suitable type of linear imager). Spectrometers 34 utilizing a folded optical system may facilitate creating an overall compact fluid analysis system 10. In some configurations, the spectrometer 34 may further include optics (e.g., one or more lenses) for collimating light from the surface 14 and/o rother suitable optics. In some example spectrometers 34, the spectrometer 34 may be configured to sense wavelengths over a range of about 390 nm to about 950 nm. Other suitable configurations of spectrometers 34 are contemplated.

The controller 18 may be coupled to one or more other electronic components of the fluid analysis system 10. For example, the controller 18 may be communicatively coupled with one or more of the illumination component 12, the light collection component 16 (e.g., the spectrometer 34 and/or other components of the light collection component 16), the motor 20, and/or one or more other suitable components of the fluid analysis system 10 and/or remote components (e.g., servers, mobile devices, etc.) that may or may not be part of the fluid analysis system 10. In some examples, the controller 18 may be configured to receive an indication to initiate a fluid analysis test (e.g., from a user via user interface or in communication with the controller 18) and send coordinated control signals to the motor 20, the one or more light sources 30, and the spectrometer 34 or other light collector to initiate movement of the motor 20 to adjust a location of the CSA 24 relative to the illumination component 12 and the collection lens system 36, to initiate illumination of a target area on the CSA 24, and initiate sensing wavelengths of light from the reactant array 26 or other suitable target area of the CSA 24.

The controller 18 may be configured to identify or may facilitate identifying a component of fluid in contact with the CSA 24 (e.g., including the surface 14) based on measured (e.g., sensed and/or calculated) levels of wavelengths of light collected from the CSA 24 with the light collection component 16 (e.g., via the spectrometer 34 and/or other suitable light collector). In some examples, the controller 18 may be configured to identify the component of fluid in contact with the CSA 24 based on one or both of a timing of the levels of the wavelength of light reflected off of the CSA 24 and an absolute change between a level of a wavelength of light collected from the surface at a time of or prior to an application of the fluid to the CSA 24 and at a predetermined time after initially applying the fluid to the CSA 24. The controller 18 may be configured to identify the component of the fluid in contact with the CSA 24 in one or more additional or alternative manners.

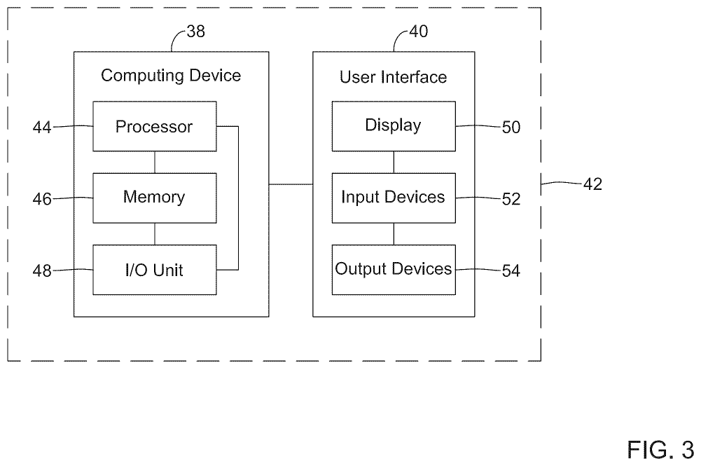

The controller 18 and/or other components of the fluid analysis system 10 may be or may include one or more computing devices including or coupled with one or more user interfaces. FIG. 3 depicts a schematic diagram of an illustrative computing device 38 and a user interface 40, where the computing device 38 and/or the user interface 40 may be entirely or partially housed in one or more housings 42 (e.g., a housing which may or may not house other components of the fluid analysis system 10). The housing 42 may be an optional component, as represented by the broken lines defining the housing 42 depicted in FIG. 3. Although various components are depicted as being included in the computing device 38 and the user interface 40, one or more of the depicted components may be omitted and/or one or more additional or alternative components may be utilized.

The computing device 38 may be any suitable computing device configured to process data of or for the fluid analysis system 10 and may be configured to facilitate operation of the fluid analysis system 10. The computing device 38, in some cases, may be configured to control operation of the fluid analysis system 10 by establishing and/or outputting control signals to the illumination component 12, the light collection component 16, the motor 20, and/or other electronic components of the fluid analysis system 10 to run a fluid analysis test and/or monitor results of a fluid analysis test. In some examples, the computing device 38 may be part of the controller 18 and may communicate with other components over a wired or wireless connection, but other suitable configurations are contemplated. When the computing device 38, or at least a part of the computing device 38, is a component separate from a structure of the controller 18, the computing device 38 may communicate with electronic components of the fluid analysis system 10 over one or more wired or wireless connections or networks (e.g., LANs and/or WANs). In some cases, the computing device 38 may communicate with a remote server or other suitable computing device.

The illustrative computing device 38 may include, among other suitable components, one or more processors 44, memory 46, and/or one or more I/O units 48. Example other suitable components of the computing device 38 that are not specifically depicted in FIG. 3 may include, but are not limited to, communication components, a touch screen, selectable buttons, and/or other suitable components of a controller. As discussed, one or more components of the computing device 38 may be separate from the controller 18 and/or incorporated into the components of the controller 18.

The processor 44 of the computing device 38 may include a single processor or more than one processor working individually or with one another. The processor 44 may be configured to receive and execute instructions, including instructions that may be loaded into the memory 46 and/or other suitable memory. Example components of the processor 44 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 46 of the computing device 38 may include a single memory component or more than one memory component each working individually or with one another. Example types of memory 46 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 46 may be or may include a non-transitory computer readable medium. The memory 46 may include instructions stored in a transitory state and/or a non-transitory state on a computer readable medium that may be executable by the processor 44 to cause the processor 44 to perform one or more of the methods and/or techniques described herein. Further, in some cases, the memory 46 and/or other suitable memory may store data received from the spectrometer 34, the motor 20, light sources 30, and/or other components of or in communication with the fluid analysis system 10.

The I/O units 48 of the computing device 38 may include a single I/O component or more than one I/O component each working individually or with one another. Example I/O units 48 may be or may include any suitable types of communication hardware and/or software including, but not limited to, communication components or ports configured to communicate with electronic components of the fluid analysis system 10 and/or with other suitable computing devices or systems. Example types of I/O units 48 may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOH protocol components, Near-Field Communication (NFC) protocol components, WI-FI protocol components, optical communication components, ZIGBEE protocol components, and/or other suitable wireless communication components), and/or other suitable I/O units 48.

The user interface 40 may be configured to communicate with the computing device 38 via one or more wired or wireless connections. The user interface 40 may include one or more display devices 50, one or more input devices 52, one or more output devices 54, and/or one or more other suitable features. In some examples, the user interface 40 may be part of or may include the computing device 38.

The display 50 may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) 52 may be and/or may include any suitable components and/or features for receiving user input via the user interface 40. Example input device(s) 52 may include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components) and/or other suitable input devices. In one example, the input devices 52 may include a touch screen that allows for setting set points, initiating a fluid analysis test, adjusting between screens (e.g., a testing screen, a data analysis screen, a results screen, etc.) and/or allows for taking one or more other suitable actions.

The output device(s) 54 may be and/or may include any suitable components and/or features for providing information and/or data to users and/or other computing components. Example output device(s) 54 include, but are not limited to, displays, speakers, vibration systems, tactile feedback systems, optical outputs, and/or other suitable output devices.

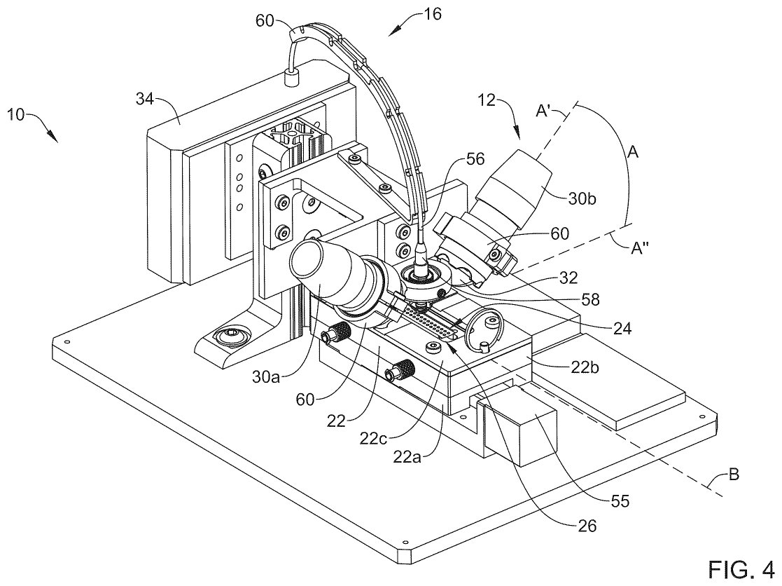

FIG. 4 depicts a schematic perspective view of an illustrative configuration of the fluid analysis system 10, where the fluid analysis system 10 may be configured to receive a CSA 24 with a linear reactant array 26. The illustrative configuration of the fluid analysis system 10 depicted in FIG. 4 may further include the illumination component 12, the light collection component 16, the adjustable stage 22, and/or other suitable components. Further, components of the fluid analysis system 10 depicted in FIG. 4 and not discussed here may optionally be omitted and/or other components not depicted in FIG. 4 may be included, as desired. In one example, although the controller 18 is not depicted in FIG. 4 as being part of the fluid analysis system 10, the fluid analysis system 10 may include or may be configured to couple with and/or otherwise communicate with the fluid analysis system 10 as discussed herein and/or in other suitable manners.

The illustrative configuration of the fluid analysis system 10 may include the motor 20 (e.g., not shown in FIG. 4, but represented by a motor housing 55 in FIG. 4) configured to drive or otherwise translate the adjustable stage 22 in opposing directions along axis B. When the reactant array 26 of the CSA 24 includes a plurality of rows and columns, the motor 20 and associated gearing may be configured to adjust the adjustable stage 22, and thus the CSA 24, in one or more directions transverse to the axis B (e.g., a direction perpendicular to the axis B and/or other suitable directions).

The illustrative configuration of the fluid analysis system 10 may or may not include a housing and may be configured to be a handheld fluid analysis system 10. To facilitate the handheld nature of a configuration of the fluid analysis system 10, the housing and/or other components of the fluid analysis system 10 may be made from light weight materials and the housing may be configured to house a battery and/or other components that may facilitate powering the fluid analysis system 10. Alternatively, the illustrative configuration of the fluid analysis system 10 may or may not include a housing and may be configured to be a bench top fluid analysis system 10.

The adjustable stage 22 may take on any suitable configuration configured to support the CSA 24 and may have one or more components. In some examples, the adjustable stage 22 may have a first component 22a, which may be a base that is configured to engage a gear or be driven in one more suitable manners, a second component 22b, which may be a body that is configured to receive and/or align the CSA 24 (e.g., in a recess thereof and/or in one or more other suitable manners), and a third component 22c, which may be a cover that is configured to facilitate maintaining a position of the CSA 24 at or within the adjustable stage 22 (e.g., by engaging a recess of the second component and/or in one more other suitable manners). One or more of the first component 22a, the second component 22b, and the third component 22c may be sub-components or portions of a single component or may be components that may be engaged with one another to form the adjustable stage 22. In some instances, the first component 22a, the second component 22b, the third component 22c, and/or other or all component of the adjustable stage 22 may be or may be part of the CSA 24 and/or the CSA 24 may be part of the adjustable stage 22. When two or more of the first component 22a, the second component 22b, and the third component 22c are components engageable together to form at least part of the adjustable stage 22, two or more of the first component 22a, the second component 22b, and the third component 22c may be coupled together in one or more suitable manners. For example, the first component 22a, the second component 22b, and the third component 22c may be coupled together using one or more threaded components (e.g., screws, etc.), one or more pins, one or more snap connections, one or more friction connections, one or more adhesives, one or more welds, and/or using one or more other suitable coupling and/or alignment techniques. In some cases, a coupling technique for coupling the second component 22b with the third component 22c may be reversible to facilitate separating the second component 22b and the third component 22c for inserting and/or removing the CSA 24 from the adjustable stage 22. Other suitable configurations of the adjustable stage 22 are contemplated.

The illumination component 12 of the illustrative configuration of the fluid analysis system 10 depicted in FIG. 4 may include a first light source 30a and a second light source 30b. As depicted in FIG. 4, the first light source 30a and the second light source 30b may be at an angle A with a surface of the CSA 24 supporting the reactant array 26 (e.g., a surface of the substrate 28 and/or other suitable surfaces). Angle A may extend between line A′ representing an axis through an individual light source 30 (e.g., the first light source 30a, the second light source 30b, etc.) and line A″ representing a line that is parallel to the surface of the CSA 24 supporting the reactants of the reactant array 26. The angle A may be any suitable acute angle, such as 45 degrees or other suitable angle.

As discussed, the light sources 30 may be at any suitable location relative to one another and relative to the CSA 24 that is configured to illuminate a target area of the CSA 24. When the light sources 30 are configured to be stationary or at a fixed location relative to an entirety of or at least a portion of the light collection component 16, the target area of the CSA 24 may be whichever portion of CSA 24 (e.g., a location of one or more reactants of the reactant array 26) has been translated to a location illuminated by the light sources 30. As depicted in FIG. 4, the first light source 30a and the second light source 30b may oppose one another, but other suitable configurations are contemplated.

Each of the first light source 30a and the second light source 30b may include the illumination lens system 32 (only the second light source 30b is depicted in FIG. 4 with the illumination lens system 32 due to an angle of the view of the fluid analysis system 10). The illumination lens system 32 may be configured to focus illumination from the respective light source 30 to the target area on the CSA 24 (e.g., on one or more reactants of the reactant array 26).

The light collection component 16 of the configuration of the fluid analysis system 10 depicted in FIG. 4 may further include the spectrometer 34, an optical fiber 56 and a lens housing 58 configured to house the collection lens system 36 (not depicted in FIG. 4) that includes one or more lenses configured to facilitate collecting light from the CSA 24. In some configurations, the optical fiber 56 may be configured to extend from the lens housing 58 to the spectrometer 34 and guide light collected at the collection lens system 36 (e.g., from the CSA 24 and/or other suitable surface 14) to the spectrometer 34.

The fluid analysis system 10 depicted in FIG. 4 may include one or more supports 60. For example, the one or more supports 60 may include one or more supports 60 configured to support the light source 30 (e.g., the first light source 30a and the second light source 30b), support the optical fiber 56 between the spectrometer 34 and the collection lens housing 58, support the collection lens housing 58 relative to the CSA 24, and/or support one or more additional and/or alternative components of the fluid analysis system 10 relative to other components thereof.

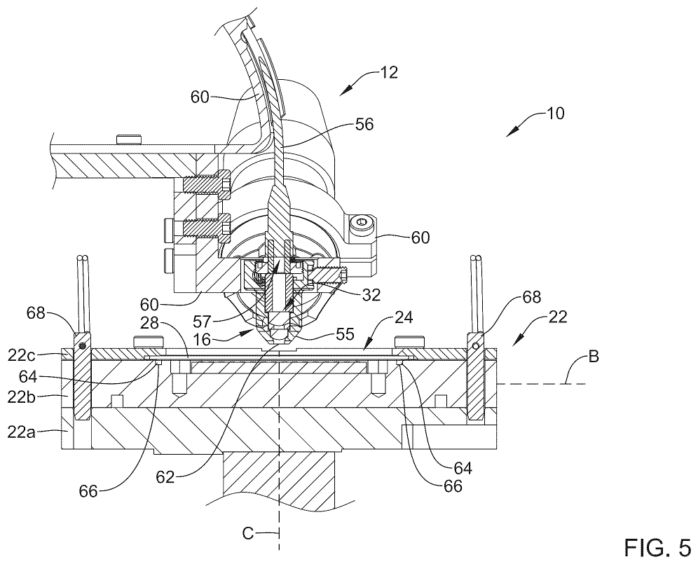

FIG. 5 depicts a schematic cross-section view of a portion of the illustrative configuration of the fluid analysis system 10 depicted in FIG. 4, taken along axis B. As depicted FIG. 5, the collection lens system 36 within the collection lens housing 58 defining an aperture 62 and a distal end 57 of the optical fiber 56 may be oriented about axis C, which may be perpendicular to or substantially perpendicular to a surface of the substrate 28 of the CSA 24 facing the collection lens housing 58 and parallel to the axis B, but other suitable configurations and/or angles between the collection lens system 36 and the surface of the CSA 24 on which the reactant array 26 is supported are contemplated.

As depicted in FIG. 5, the adjustable stage 22 may be configured to receive the CSA 24 between the second component 22b and the third component 22c, where the third component 22c may cover a portion of the CSA 24 and define an opening through which the light from a target area on the CSA 24 may pass to the light collection component 16 (e.g., to the collection lens system 36). In operation, the third component 22c may be removed from the second component 22b and the CSA 24 may be positioned onto the second component 22b. In some examples, the CSA 24 may include one or more locator pins 64 configured to engage one or more locator openings 66 of the second component 22b to facilitate proper alignment of the CSA 24 relative to the adjustable stage 22 and/or the light collection component 16, but other suitable alignment configurations may be utilized or alignment configurations for the CSA 24 may be omitted. Once the CSA 24 is positioned on or in the second component 22b, the third component 22c of the adjustable stage 22 may be placed on top of the second component 22b so as to cover a portion of the CSA 24 and secure the CSA 24 within the adjustable stage 22. At least the second component 22b and the third component 22c may be coupled to one another with one or more connector pins 68 (e.g., two connector pins 68, as depicted in FIG. 5) and/or one or more other suitable components.

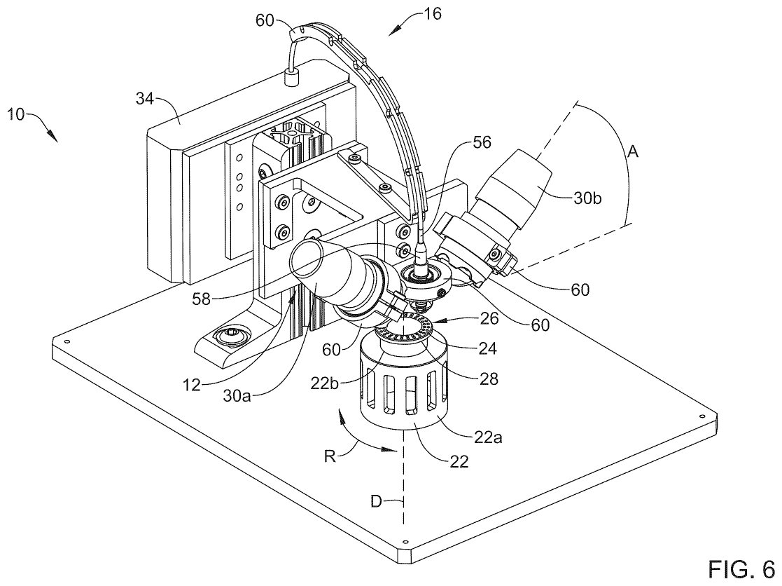

FIG. 6 depicts a schematic perspective view of an illustrative configuration of the fluid analysis system 10, where the fluid analysis system 10 may be configured to receive a CSA 24 with an annular reactant array 26. The illustrative configuration of the fluid analysis system 10 depicted in FIG. 6 may further include the illumination component 12, the light collection component 16, and the adjustable stage 22. The illumination component 12 and the light collection component 16 may be the same as or similar to the configurations of the illumination component 12 and the light collection component 16 of the illustrative configuration of the fluid analysis system 10 depicted in FIG. 4. Further, components of the fluid analysis system 10 depicted in FIG. 6 and not discussed herein may optionally be omitted and/or other components not depicted in FIG. 6 may be included, as desired. In one example, although the controller 18 is not depicted in FIG. 4 as being part of the fluid analysis system 10, the fluid analysis system 10 may include or may be configured to couple with and/or otherwise communicate with the fluid analysis system 10 as discussed herein and/or in other suitable manners.

Although not depicted in FIG. 6, the illustrative configuration of the fluid analysis system 10 may include the motor 20, which may be configured to drive the adjustable stage 22 in one or more rotational directions R about an axis D that may be offset from and perpendicular with axis C about which the illumination lens configuration may be positioned (see FIG. 5). Further, the motor 20 may be configured to cause the adjustable stage 22 to adjust in one or more other suitable manners including, but not limited to, by causing the adjustable stage 22 to rotate about one or more axes in addition to or as an alternative to axis D, by causing the adjustable stage 22 to adjust laterally (e.g., adjust radially inward or radially outward), and/or by causing the adjustable stage 22 to adjust relative to the illumination component 12 and/or the light collection component 16 in one or more other suitable manners. In some examples, when the annular reactant array 26 includes two or more rings of reactants and/or in other instances, the motor 20 may be configured to adjust the CSA 24 in a radially outward and/or radially inward direction (e.g., in a linear direction toward and/or away from axis C) in addition to rotationally about axis D. In some examples, when the annular reactant array 26 includes a spiral pattern of reactants and/or in other instances, the motor 20 may be configured to adjust the CSA 24 radially outward and/or radially inward simultaneously or iteratively with the rotational adjustment of the CSA 24.

The adjustable stage 22 of the fluid analysis system 10 depicted in FIG. 6 may take on a cylindrical shape, a shaft shape, a rod shape, an axle shape, and/or other suitable shape configured to facilitate rotating the CSA 24. The illustrative adjustable stage 22 may have a first component 22a, which may be a base configured to engage a gear (e.g., a worm gear, etc.) or be driven in one more suitable manners and a second component 22b, which may be an extension portion configured to extend from the first component 22a and support the CSA 24. The first component 22a and the second component 22b may be sub-components or portions of a single component or may be two components that may be engaged to form the adjustable stage 22. Other suitable configurations of the adjustable stage 22 are contemplated.

The adjustable stage 22 may be configured to engage the CSA 24 with the annular reactant array 26 in any suitable manner. For example, the CSA 24 with the annular reactant array 26 may engage the adjustable stage 22 using one or more adhesives, one or more magnetic connections, one or more snap connections, one or more friction fit connections, and/or one or more other suitable types of connections.

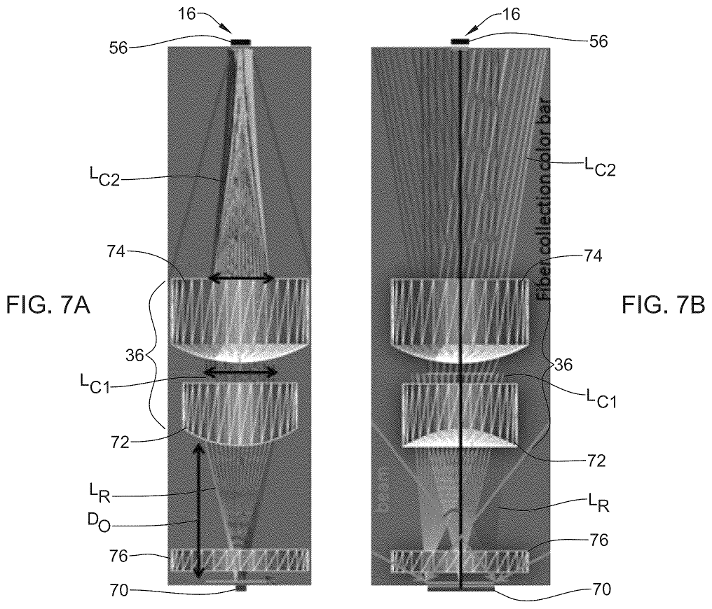

FIGS. 7A and 7B depict schematic views of an illustrative collection lens system 36 relative to a reactant 70 of the reactant array 26 and the optical fiber 56 of the light collection component 16, where the reactant 70 has elongated bar configuration (e.g., where the reactant 70 may be the target area or at least a portion of the target area of the CSA 24). FIG. 7A depicts a schematic view of the illustrative collection lens system 36 along a first plane. FIG. 7B depicts a schematic view of the illustrative collection lens system 36 along a second plane perpendicular to the first plane of the view depicted in FIG. 7A.

In some examples of the illustrative collection lens system 36, the collection lens configuration may include, but is not limited to, a focusing lens 72 and an imaging lens 74 that may work together to optically guide light from the reactant(s) 70 having a first shape or configuration (e.g., a line, rectangle, circle, etc.) to the optical fiber 56 or other light collector component having a second shape or configuration (e.g., a line, a rectangle, a circle, etc.) that may be the same as or different than the first shape or configuration. As such, the lens(es) of the collection lens system 36 may be configured and/or optimized for a shape and/or size of a target area or reactant 70 of the reactant array 26 and a shape and/or size of the optical fiber 56 or other suitable light collector component.

Any suitable focusing lens 72 may be utilized including, but not limited to, a cylinder lens (e.g., a lens having different radii in an x and y axis) and/or other suitable lens selected to focus light from the reactant 70. Any suitable imaging lens 74 may be utilized including, but not limited to, a spherical lens, an aspheric lens, and/or other suitable lens configured to focus light from the focusing lens 72 at a desired location (e.g., at the optical fiber 56 and/or other suitable light collector component).

In some examples, the focusing lens 72 may have a focusing power for collimating light L_{R} from the reactant 70 into partially collimated light L_{C1}, as may be viewed from along the first plane perspective in FIG. 7A. The partially collimated light L_{C1} from the focusing lens 72 may not be fully focused because the focusing lens 72 may not have a focusing power for light in the second plane and as such, the imaging lens 74 may bend the partially collimated light L_{C1} into fully or nearly fully collimated light L_{C2}. As a result, the light L_{R} from reactant 70 may be directed to and/or collected by the optical fiber 56 in configurations where the reactant 70 may have a different shape than a shape of the optical fiber 56. Further, such a configuration of the collection lens system 36 where the focusing lens 72 at least partially collimates light from an entirety of the reactant 70 or target area may result in automatically averaging of the light collected from the entire reactant 70 or target area without requiring adjustment of the CSA 24 relative to the illumination component 12 and/or the light collection component 16.

In one example configuration of the collection lens system 36 relative to the reactant 70 and the optical fiber 56, the reactant 70 may be rectangular, the focusing lens 72 may be a cylinder lens, the imaging lens 74 may be an aspheric lens, and the optical fiber 56 may have a circular circumference and/or cross-section. In the example, the reactant 70 may have a width of 0.5 mm and a length of 3.0 mm. Optionally, a transparent intermediary 76 (e.g., a glass slide, etc.) may be positioned between the reactant 70 and the focusing lens 72, where the transparent intermediary 76 may have a thickness of 1 mm with a 0.5 mm gas flow space between the reactant 70 and the transparent intermediary 76. There may be a 6.5 mm object distance Do between the reactant 70 and the focusing lens 72, which may facilitate isolating fluid to be analyzed proximate the CSA 24 and/or prevent contamination of the reactant array 26. There may be a 3.0 mm diameter (or smaller, such as 2.0 mm diameter and/or other suitable size) aperture between the focusing lens 72 and the imaging lens 74. The optical fiber 56 may have a 0.8 mm diameter. There may be an 11 mm focus distance between the imaging lens 74 and the optical fiber 56. Although illustrative sizes for the setup in the example are provided, other suitable sizes and/or components of the collection lens system 36 are contemplated for various configurations of reactants 70, light collector components, and/or the components of the fluid analysis system 10.

To focus light from the light source 30 on one or more reactants of the reactant array 26, the fluid analysis system 10 may include the illumination lens system 32. The illumination lens configuration may have any suitable set of one or more lenses. In some example configurations of the illumination lens system 32, the lens configuration of the illumination lens system 32 may be the same as or similar to the collection lens system 36 and may utilize a focusing lens 72 and an imaging lens 74. For example, the imaging lens 74 (e.g., a spherical lens, an aspheric lens, and/or other suitable imaging lens 74) may be proximate the light source 30 which may provide light from an area having a first shape or configuration (e.g., a circular spot area of an LED, or a circular spot area of an optical fiber providing light to the reactant(s), and/or other shape or configuration) to the focusing lens 72 (e.g., a cylinder lens and/or other suitable focusing lens 72) that may be located between the imaging lens 74 and a target area of the CSA 24 (e.g., where the target area may be or may include one or more reactants of the reactant array 26) that has a second shape or configuration (e.g., a line, a rectangle, a circle, etc.) that is the same as or different than the first shape or configuration. The matching of the shape of the light provided to the target area of the CSA 24 from the light source 30 may result in a natural averaging of the light along the reactant at the target area of the CSA 24, which facilitates analyzing a single reactant of the reactant array 26 without movement of the CSA 24 relative to the illumination component 12 and/or the light collection component 16. Other suitable sizes and/or components for the illumination lens system 32 are contemplated for various configurations of reactants 70, light sources 30, and/or the other components of the fluid analysis system 10.

Other suitable lens configurations are contemplated. For example, one or more lens configurations may be configured to have an adjustable focal length and/or may have a zoom lens configuration. Lens configurations with adjustable focal lengths and/or zoom configurations may facilitate intentionally blurring light sensed or collected from a target area, focusing on different planes when a surface of interest is not level and/or is not at a predetermined depth, and/or facilitate capturing light in one or more other suitable manners.

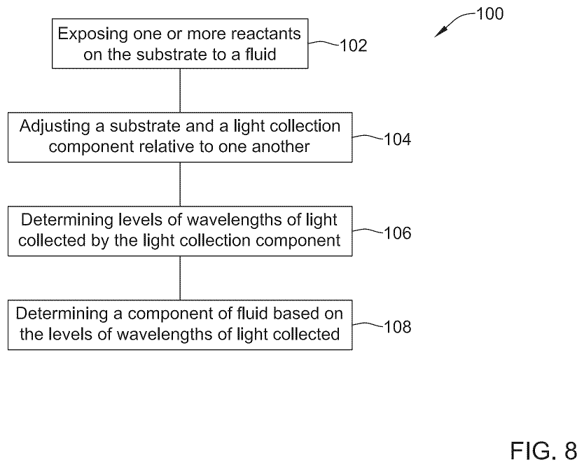

FIG. 8 depicts a schematic diagram of an illustrative method 100 for analyzing a fluid (e.g., an illustrative method for performing a fluid analysis test) using the fluid analysis system 10 and/or other suitable system, where the method 100 may be used to determine the fluid and/or one or more components of the fluid. The fluid and/or the component of the fluid that is to be determined may be an analyte of interest and one or more reactant of the reactant array 26 may be configured to be sensitive to the analyte.

As depicted in FIG. 8, the method 100 may include exposing 102 one or more reactants of the CSA 24 (e.g., one or more reactants of the reactant array 26 on the substrate 28 and/or other suitable reactants) to one or more fluids. The reactants of the CSA 24 may be exposed to the fluid in any suitable manner. For example, exposing the reactants of the CSA 24 to the fluid may include pumping the fluid along the reactant array 26 (e.g., between the reactant array 26 and a cover (e.g., the at least partially transparent intermediary 76 and/or other suitable cover) of or within a housing of the fluid analysis system 10, pumping the fluid along the reactant array 26 within a cartridge, passively applying fluid to the reactant array 26 (e.g., without actively pumping the fluid along the reactant array 26), and/or exposing one or more of the reactants of the CSA 24 to the fluid in one or more other suitable manners.

The method 100 may include adjusting 104 one or more of the CSA 24 (e.g., the substrate 28 of the CSA 24), the illumination component 12, the light collection component 16, and/or one or more other suitable components relative to one another to facilitate collecting light from one or more of the reactants of the CSA 24. In some examples, the motor 20 may be actuated to adjust a location of the adjustable stage 22 and the substrate 28 relative to the light collection component 16 (e.g., relative to the collection lens system 36 and/or the optical fiber 56 collecting light from the reactants). Adjusting the substrate 28 relative to the light collection component 16 may facilitate allowing the light collection component 16 to collect light from one or more of the reactants of the reactant array 26 individually as each reactant passes a target area at which light from the illumination component 12 is focused and at which light is collected from the CSA 24. In some examples, the target area may be located, sized, and/or shaped to align with reactants as the substrate 28 is adjusted.

The adjusting of the CSA 24 relative to the light collection component 16 may occur before, during, and/or after exposing the one or more reactants of the CSA 24 to the fluid, as desired. In some examples, the adjusting of the CSA 24 relative to the light collection component 16 may occur during and/or after exposure of the one or more reactants to the fluid. The adjusting of the CSA 24 relative to the light collection component 16 may be continuous during a fluid analysis test and/or at one or more suitable intervals during the fluid analysis test.

The CSA 24 may be adjusted relative to the target area during a fluid analysis test such that one or more reactants of the reactant array 26 pass the target area one or more times during the fluid analysis test. For example, during a fluid analysis test, the CSA 24 may be adjusted such that one or more of the reactants of the reactant array 26 pass the target area one time, two times, three times, five times, ten times, twenty times, and/or pass the target area one or more other suitable number of times during the fluid analysis test. In one example, during a fluid analysis test the CSA 24 may be adjusted relative to the target area such that all of the reactants of the reactant array 26 pass the target area ten times. In another example, during a fluid analysis test the CSA 24 may be adjusted relative to the target area such that a predetermined subset of (e.g., a predetermined number of) reactants of the reactant array 26 pass the target area a plurality of times. Other suitable adjustments of the CSA 24 during a fluid analysis test are contemplated.

Light may be applied to the CSA 24 (e.g., to the substrate 28 of the CSA 24) from the illumination component 12 to facilitate collecting light from the reactants of the reactant array 26 during a fluid analysis test. In some examples and as discussed, the light applied to the CSA 24 may form or at least partially form the target area on the substrate 28 at which the light collection component 16 may be focused for collecting light.

Any suitable configuration of light sources, as discussed herein or otherwise, may be utilized for applying light to the CSA 24. In some examples, as discussed herein, light may be applied to the substrate from a first light source at a first angle and a first location with respect to the substrate 28 of the CSA 24 and a second light source at a second angle and a second location with respect to the substrate 28, where the first and second angles may be the same and the second location may be different than the first location. Other suitable configurations of the light sources of the illumination component 12 are contemplated.

Light from reactant array 26 may travel to a light collector of the light collection component 16, such as the spectrometer 34, and the light collector may determine an amount of each wavelength of light collected by the light collection component 16 over a predetermined range of wavelengths (e.g., over the wavelengths of the visible light spectrum, over wavelengths in a spectrum of about 425 nm to about 725 nm, and/or other suitable ranges or spectra). The light from the reactant array 26 and/or from other suitable locations of the CSA 24 may be collected and measured continuously during a fluid analysis test and/or at one or more suitable intervals (e.g., when a reactant of the reactant array is positioned at a target area of the illumination component 12 and/or the light collection component 16). Data related to the collection and measurement of the light from the reactant array 26 may be time stamped and/or location stamped (e.g., relative to the fluid analysis test) and used for analysis and/or stored for later analysis.

The amount of each wavelength of light collected by the light collection component 16 from the reactant array 26 may be a count of photons, light intensity amounts (e.g., candela/steradian), and/or other suitable measurements of light amounts at the different relevant wavelengths. In some cases, the wavelengths over the spectrum of interest may be grouped or binned with adjacent wavelengths (e.g., where each bin may include two wavelengths, three wavelengths, four wavelengths, five wavelengths, ten wavelengths, etc.) and the amount of wavelengths of light for the wavelengths of each bin may be averaged to reduce a number of data points collected to a manageable amount, but this is not required.

The light collector (e.g., the spectrometer 34 and/or other suitable light collector) and/or the controller 18 may determine 106 a level of wavelengths of light collected by the light collection component 16 from each reactant of the reactant array 26 passing through the target area. In some instances, the determining 106 of the level of the wavelengths of light collected by the light collection component 16 may include determining the levels of the wavelengths of light collected from each reactant of the reactant array 26 over time at multiple instances or intervals during a fluid analysis test.

The determined levels of the wavelengths of light collected may be any suitable measurements or values related to the collected light. In some examples, the level of the wavelengths of light collected by the light collection component 16 may be an amount or quantity of wavelengths of light measured by the light collector (e.g., a number of photons of each wavelength collected, an intensity of each wavelength detected, etc.) for each of one or more reactants of the reactant array 26. Alternatively or additionally, in some examples, the level of wavelengths of light collected by the light collection component 16 may be a normalized value of the amount of the wavelengths of light collected by the light collector for each of one or more reactants of the reactant array 26 and/or one or more other suitable values, as desired. The normalized value of the amount of the wavelengths of light collected may be referred to as a “reflectance” calculation, level, value, or measurement, but this is not required.

The normalized value of an amount or quantity of the wavelengths of light collected may be determined based on calculating amounts of the wavelengths of light associated with one or more reference spectra. In some examples, one of the reference spectra may be a dark reference spectra and another of the reference spectra may be a light reference spectra (e.g., a “100% white” reference spectra). The following equation represents an illustrative normalized value of an amount of a wavelength of light collected by the light collection component 16:

$${\text{Normalized Value of an amount of a Wavelength}}_{n} \text{of Light} = ( {( \text{sampled photon count} )}_{n} - ( \text{dark photon count} ) ) / ( ( \text{light photon count} ) - ( \text{dark photon count} ) ) \qquad ( 1 )$$

where, n is the wavelength or wavelength bin or wavelength spectrum at which the sampled photon count was taken. When such a normalization is applied to each wavelength quantity data, the resulting value may be independent of a variation in spectral response of the fluid analysis system 10 or any intended or unintended spectral filtering by the illumination lens system 32, the collection lens system 36, and/or other components of the fluid analysis system 10. Other suitable techniques for normalizing collected data are contemplated. In some cases, the normalized value of an amount of a wavelength of light (or a spectrum of wavelengths) may be considered a value of reflectivity and expressed as a percentage of a total amount of light that may be sensed or collected at the light collection component 16.

The dark reference spectra (e.g., the dark photon count in equation (1)) may be or may include amounts of wavelengths of light collected by the light collection component 16 from the CSA 24 and measured by the spectrometer 34 or other suitable light collector when illumination of the CSA 24 is completely extinguished and ambient light is blocked. Alternatively or additionally, the dark reference spectra may be collected when ambient light is present as long as the light from the light collection component 16 is not saturated and meets an acceptable amount of range for sensor elements of the light collection component 16 (e.g., less than 25% of an entire dynamic range for the sensor elements and/or other suitable amount of range for the sensor elements).

When the substrate 28 of the CSA 24 is formed from one or more materials having a white surface (e.g., a porous white plastic material that has a high (e.g., as high as 100% or about 100%) diffuse reflectivity over an entire visible spectrum), the illumination component 12 may be initiated to illuminate a white, non-reactant portion of the CSA 24 and the light collection component 16 may capture light from the illuminated white, non-reactant portion of the CSA 24. The light from the white, non-reactant portion of the CSA 24 that is captured by the light collection component 16 may be measured by the spectrometer 34 or other suitable light collector and stored as the light reference spectra (e.g., the light photon count in equation (1)).

A sample spectra measurement from reactants on the CSA 24 (e.g., the sampled photon count in equation (1)) may be or may include amounts of wavelengths of light collected by the light collection component 16 from the reactants of the CSA 24. In some examples, the sample spectra measurements from the reactants may include amounts of wavelengths of light collected when the CSA 24 is positioned such that illumination from the illumination component 12 is illuminating a reactant of the reactant array 26 and the light collection component 16 is collecting light from the illuminated reactant.

When the surface of the substrate 28 of the CSA 24 is not a perfect 100% diffuse reflector, a correction may be made to the light reference spectra prior to using the light reference spectra in equation (1). Further, when the response of the spectrometer 34 and/or other suitable light collector is non-linear with respect to amplitude, mathematical corrections may be applied, as desired, to the sample spectra, the dark reference spectra, and/or the light reference spectra.

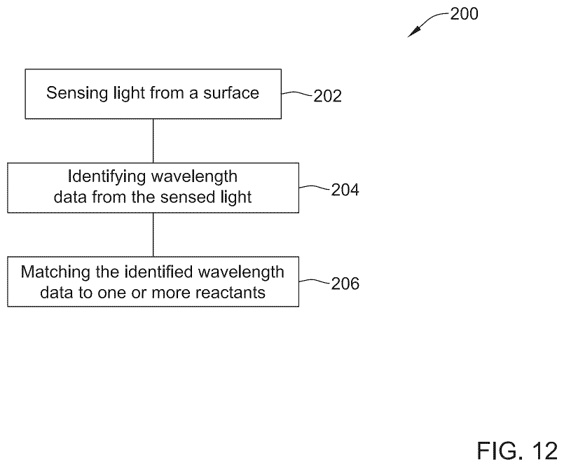

In some instances, the levels of the wavelengths of light collected by the light collection component 16 may not be directly associated with a reactant of the reactant array. As a result, the levels of the wavelengths of light collected by the light collection component 16 may be associated with one or more reactants of the reactant array 26 based on, for example, a known time of when a test started, a known rate at which the CSA is adjusted, and when the light was collected from the CSA 24. In another example, the levels of the wavelengths of light collected by the light collection component 16 may be associated with reactants of the reactant array 26 by running a data analysis (e.g., one or more algorithms) on data related to the collected light and then assigning or associating the levels of the wavelengths of light collected by the light collection component 16 with one or more reactants of the reactant array 26 based on the data analysis. Additional and/or alternative configurations for assigning or associating collected light and related data with one or more reactants of the reactant array 26 may be utilized. Assigning or associating levels of wavelengths of light to one or more reactants is discussed further with respect to FIGS. 12-15.

As depicted in FIG. 8, the method 100 may include determining 108 a component of the fluid sample to which the CSA 24 is exposed based on the levels (e.g., amounts) of the wavelengths of light collected by the light collection component 16. In some examples, one or more sets of data based on the levels of wavelengths of light collected by the light collection component 16 for one or more reactants of the reactant array 26 may be determined and compared to known data associated with one more fluids or components of fluids (e.g., predetermined sets of data of or related to levels of wavelengths of light for one or more reactants of the reactant array 26, where each predetermined set of data is associated with a fluid or a component of fluid, such as a fluid of interest or a fluid of one or more fluids in a database). Where a known or predetermined data set associated with a fluid of the one or more fluids or component of fluids in a database matches the one or more determined sets of data for the one or more reactants of the reactant array 26 (e.g., levels of the wavelengths associated with one or more reactants, etc.), the fluid or component of the fluid associated with the known or predetermined data set may be identified and indicated as being or as being part of the fluid sample to which the CSA 24 was exposed prior to or during the fluid analysis test. Predetermined data sets may be considered to match the determined set of data when the respective data is the same, when a portion of the respective data is the same, a pattern in the data is the same, and/or in other suitable instances. The controller 18 and/or other suitable components of or in communication with fluid analysis system 10 may be configured to compare data in determined sets of data of or related to the levels of wavelengths of light to similar data in the known or predetermined data sets of or related to the levels of wavelengths of light associated with the possible components of the fluid and/or identify components of the fluid to which the reactants of the CSA 24 are exposed based on the comparison.

Any suitable data related to the levels of the wavelengths of light collected by the light collection component 16 may be determined and/or utilized to determine the fluid or component of the fluid sample. Example suitable factors include, but are not limited to, a level (e.g., an amount of light such as a photon count, a normalized value of an amount of light, or other suitable level) of wavelengths of light at one or more reactants, a change (e.g., an absolute change and/or other suitable change) in a level of wavelengths of light at one or more reactants, a change between a first predetermined time (e.g., before exposure to the fluid, at a predetermined time after exposure to the fluid, etc.) and a second predetermined time (e.g., at or after an end of the test, at a predetermined time after exposure to the fluid, etc.), a statistical analysis (e.g., a mean of, minimum of, maximum of, etc.) of levels of wavelength of light at one or more reactants, a timing of a level of wavelengths of light at one or more reactants, a timing of a change of a level of wavelengths of light at one or more reactants (e.g., at what time(s) during the fluid analysis test did the reactant change colors), a combination of one or more determined pieces of data associated with one or more reactants, and/or other suitable data. In one example, a component of a fluid sample to which the CSA 24 was exposed prior to or during a fluid analysis test using the fluid analysis system 10 may be determined based on one or both of a timing of a level of wavelengths of light collected from one or more reactants of the CSA 24 and an absolute change between a level of a wavelength of light collected from the one or more reactants of the CSA 24, where the change occurred from a time prior to a beginning of an application of fluid to the reactants of the CSA 24 and a predetermined time after initially applying the fluid to the reactants. Other suitable techniques for identifying a fluid of the component using the fluid analysis system 10 are contemplated.

In some examples, determining 108 a component of a fluid to which one or more reactants of the reactant array 26 are exposed during a fluid analysis test may include identifying or calculating statistical data (e.g., minimum, maximum, mean, etc.) for levels of the wavelengths of light collected. For example, statistical data for levels of the wavelengths of light collected by the light collection component 16 at a plurality of instances of time before, during, or after a fluid analysis test may be calculated or otherwise identified and that identified statistical data may be compared to predetermined sets of statistical data associated one or more fluids or fluid components in a database of fluids and/or fluid components to identify a fluid or a component of fluid in a fluid.

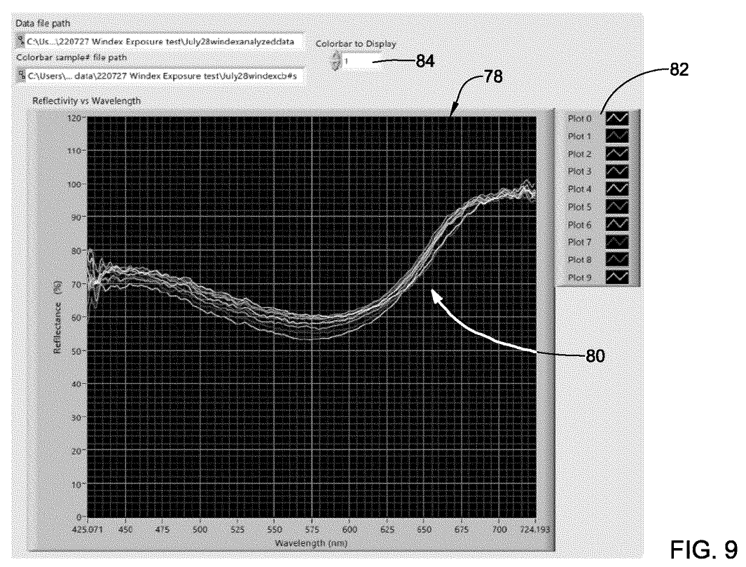

FIGS. 9-11 depict schematic graphs 78 of levels of wavelengths of light captured from three different reactants during a fluid analysis test with the fluid analysis system 10, where the reactants were continuously exposed to a fluid (e.g., a sample fluid) and passed by a target area of the illumination component 12 and the light collection component 16 ten times during exposure to the fluid. The ten passes of the reactants through the target area are represented, respectively, by plots 80 zero through nine, as indicated in the key 82 (e.g., where the different plots are color coded) adjacent the graphs 78. Collected data from one or more reactants may be used individually or together to identify one or more components of the fluid to which the reactants were exposed, as discussed herein or otherwise.

The graphs 78 of FIGS. 9-11 include a spectrum of wavelengths (nm) of interest for a light collector (e.g., the spectrometer 34) on the X-axis and a reflectance, measured as a percent (%), on the Y-axis. The reflectance may be a measure of or related to a level of wavelengths of light collected by the light collection component 16 from the reactants and may be calculated using equation (1).

Although the graphs 78 in FIGS. 9-11 depict changes in reactants during a fluid analysis test in which the reactants were continuously exposed to the fluid during the fluid analysis test and data collection, it is contemplated the fluid may be removed from reactants before or during the fluid analysis test and data collection. Further, although graphs 78 for light collected from only three of the reactants are depicted, values for each reactant may be calculated and graphed.

FIG. 9 depicts a graph 78 with ten plots 80 of the level of wavelengths of light collected from a first reactant (e.g., a reflectance of light collected from the first reactant) over a spectrum of wavelengths observed by the light collector (e.g., the spectrometer 34). An indication that the graph 78 shows values of reflectance for the first reactant may be indicated in a reactant indicator box 84 on a screen with the graph 78, but other suitable configurations are contemplated. As the plots 80 of the reflectance at each wavelength closely follow one another over a time period of the fluid analysis test (e.g., from plots 0 through 9), the first reactant of the reactant array 26 may have a minimal sensitivity to the components of the fluid to which the reactant array 26 was exposed.

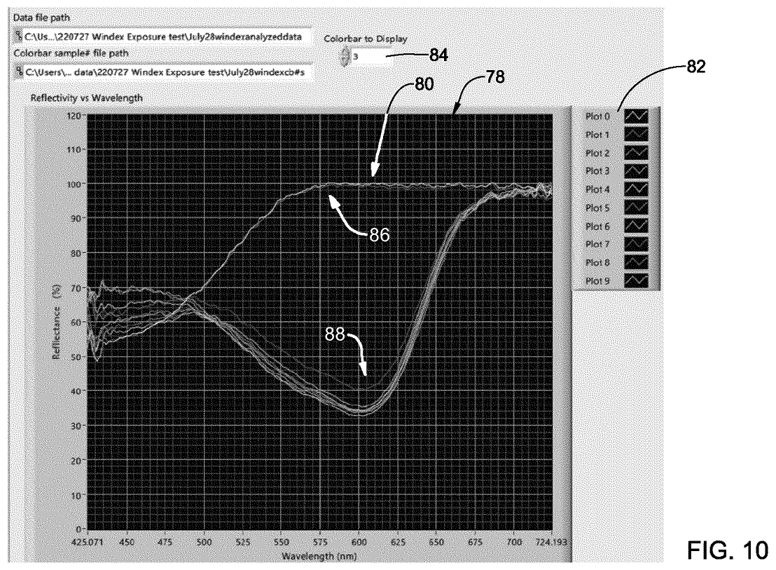

FIG. 10 depicts a graph 78 with ten plots 80 of the level of wavelengths of light collected from a third reactant (e.g., a reflectance of light collected from the third reactant) over a spectrum of wavelengths observed by the light collector (e.g., the spectrometer 34). An indication that the graph 78 shows values of reflectance for the third reactant may be indicated in the reactant indicator box 84 on a screen with the graph 78, but other suitable configurations are contemplated. As the plots 80 of the reflectance at each wavelength abruptly change paths in a range of wavelengths of about 490 nm to about 700 nm after the second plot 80 (e.g., the zero and first plots 80 generally follow a first path 86 and plots 80 two through nine generally follow a second path 88), the third reactant has a sensitivity to at least one component of the fluid to which the reactant array 26 was exposed after an exposure amount reaches or goes beyond a threshold amount of exposure to the fluid.

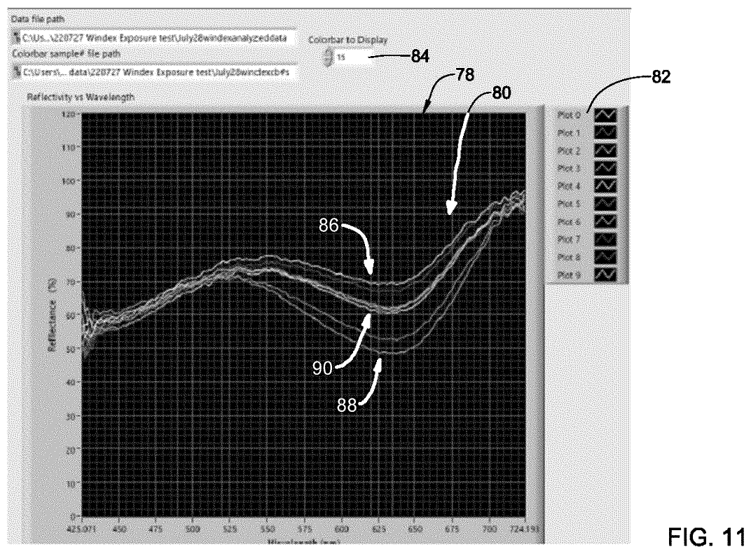

FIG. 11 depicts a graph 78 with ten plots 80 of the level of wavelengths of light collected from a fifteenth reactant (e.g., a reflectance of light collected from the fifteenth reactant) over a spectrum of wavelengths observed by the light collector (e.g., the spectrometer 34). An indication that the graph 78 shows values of reflectance for the fifteenth reactant may be indicated in the reactant indicator box 84 on a screen with the graph 78, but other suitable configurations are contemplated. As the plots 80 of the reflectance at each wavelength abruptly change paths in a range of about 530 nm to about 710 nm after the first two plots 80 and then again after the next two plots 80 (e.g., the zero and first plots 80 generally follow a first path 86, the second and third plots 80 generally follow the second path 88, and the fourth through ninth plots 80 generally follow a third path 90 between the first path 86 and the second path 88), the fifteenth reactant has a sensitivity to at least one component of the fluid to which the reactant array 26 was exposed after an exposure amount reaches or goes beyond a first threshold amount of exposure to the fluid and then the sensitivity to the at least one component of the fluid is reduced after an exposure amount reaches or goes beyond a second threshold amount of exposure to the fluid or after a set time period of exposure to the at least one component of the fluid. It is noted that the first path 86 and the second path 88 depicted in FIG. 11 are not necessarily the same paths as depicted in FIG. 10.

To ensure the fluid analysis system 10 produces accurate results, it may be desirable to ensure the light collection component 16 is centered on a reactant when collecting light from the reactant. It can be difficult and time-consuming, however, to iteratively adjust the CSA 24 or adjust components of the fluid analysis system 10 such that a center (e.g., in two dimensions) of each reactant of the reactant array 26 is centered at a target area (e.g., a focal point) of the light collection component 16 to ensure a sufficient amount of light is collected from the reactant so as to be able to make an accurate determination concerning the reactant (e.g., whether and to what extent and at what time the reactant reacted to exposure to a fluid being tested). To address such a tedious process, it may be expeditious to align reactants of the CSA 24 with the target area of the light collection component 16 in one-dimension and continuously adjust the CSA 24 along the one dimension at a constant rate throughout a fluid analysis test, while continuously collecting light from the CSA 24 at a constant rate and storing data related to the collected light. The collected light may include collected light from locations of the reactants and non-reactant locations of the CSA 24.

In some instances, as discussed, the light collector of the fluid analysis system 10 may be a spectrometer 34 with a linear array photo-detector. When used, due to how the linear array photo-detector is read-out serially by an analog-to-digital converter (ADC), the spectrometer 34 may be most accurate when collecting (or sampling) light continuously at a constant rate. As such, adjusting the CSA 24 relative to the light collection component 16 at a constant rate and continuously collecting light from the CSA 24 may result in obtaining the most accurate data possible from the light collection component 16, particularly when the light collector thereof may be the spectrometer 34.

When light data is collected continuously (e.g., continuously at a constant rate, which may or not be iteratively), however, a location on the CSA 24 from which the light data is from may not necessarily be known at the time of light collection. As such, it may not be clear which light data from the CSA 24 is associated with a reactant and/or with which reactant of the CSA 24.

In some instances, algorithms, implemented by a computing device (e.g., the controller 18 and/or other suitable computing devices 38), may be used to convert amplitude (photon count, etc.) versus wavelength data from the spectrometer 34 to a reflectivity spectra graph (e.g., a graph of normalized levels of wavelengths of light) for each repetitive sample of light collected for the fluid analysis test. The average reflectivity (e.g., the average percentage of total potential light that may be collected) for the entire spectra of a sample of light collected may then be computed and graphed versus sample number. The minima of this graph may be on the sample numbers of the optimum sample numbers for each reactant. A further algorithm may perform another pass on the sample number data comparing differences between sample numbers to assign specific optimum sample numbers to their specific reactants. Additionally, the whole process can be run repetitively while translating the linear stage alternately in the forward and reverse scan directions with an adjustable fixed delay time between the mechanical scans. Then the data can be sorted to present graphs showing precisely how the spectra change over a given sequence of time intervals, for example, as the CSA is exposed to a given fluid. FIGS. 12-15 schematically depict illustrative configurations for assigning or associating data with reactants and identifying fluid and/or components of fluid tested in a fluid analysis test.

FIG. 12 is a schematic diagram of an illustrative method 200 of matching or associating collected wavelength light data across a spectrum of light collected with one or more reactants of a reactant array 26 for a fluid analysis test. The collected wavelength data to be associated with one or more reactants may be or may include levels of wavelengths of light that are determined and/or other data determined or calculated from the collected wavelength light data, as discussed herein or otherwise.

The method 200 may include sensing 202 light from a surface (e.g., light reflected, transmitted, etc. from the surface of the CSA 24 facing the light collection component 16). Light from the surface may be sensed in any suitable manner as discussed herein or otherwise. In some examples, the light from the surface may be continuously sensed while scanning the surface during a fluid analysis test and the fluid analysis test may include scanning a predetermine length of the surface (e.g., a full length of the surface or a portion of the full length) multiple times during the fluid analysis test.

In one example of scanning the CSA 24 and sensing light from the CSA 24, the CSA 24 may be positioned relative to the light collection component 16 such that a first end of the CSA 24 is located at a target area of the light collection component. The CSA 24 may then be adjusted such that the reactants of the reactant array 26 on the CSA 24 pass the target area multiple times. When the CSA 24 is in a linear configuration, the CSA 24 may be translated back and forth along its length at a constant rate and when the CSA 24 is an annular configuration, the CSA 24 may be continuously rotated at a constant rate. The light sensed or collected may be continuously sensed or collected at every 0.1 mm of adjustment of the CSA 24 and/or at other suitable intervals to obtain wavelength data sets for a predetermined spectrum (e.g., a spectrum of wavelengths from about 400 nm to about 700 nm, etc.) In one example, fifteen sets of data may be collected per scan of the CSA 24, but other suitable number of data sets may be collected. Any suitable number of scans of the CSA 24 may be performed during a fluid analysis test and in one non-limiting example, twenty scans of the CSA 24 may be performed and three hundred data sets per scan may be identified.

From the sensed light, wavelength data may be identified 204. The wavelength data identified may be any suitable wavelength data discussed herein or otherwise including, but not limited to, levels of wavelengths of the light sensed (e.g., collected), normalized levels of wavelengths of light sensed, timing of levels of wavelengths of light sensed and/or other suitable wavelength related data. The wavelength data identified may include wavelength data for reactant portions of the surface of the CSA 24 and for non-reactant portions of the surface of the CSA 24. The wavelength data may include or be based on (e.g., may be a function of) levels of wavelength of light for a full predetermined spectrum of light (e.g., the predetermined spectrum may be set by the light collector and/or set in one or more other suitable manners). In some examples the identifying step 204 may be performed separate from the sensing step 202 and in some examples the identifying step 204 may be performed with the sensing step 202 as a single step.

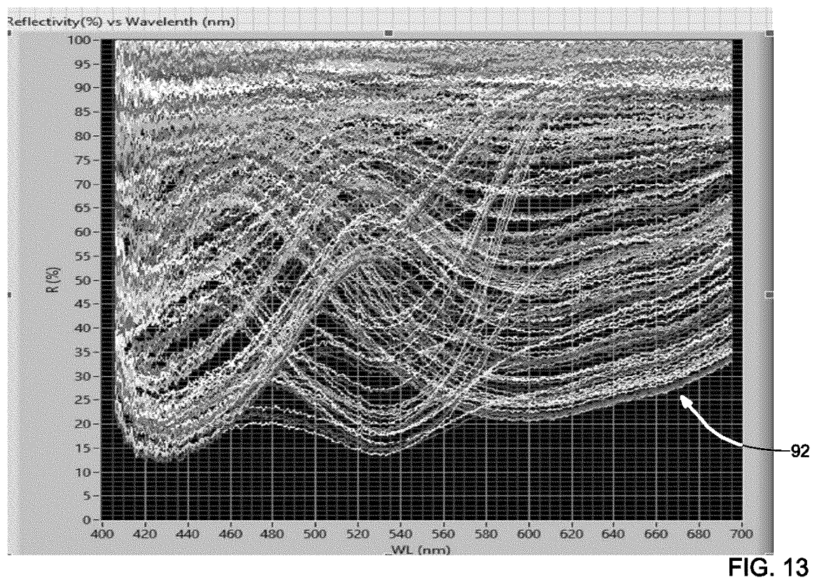

In one example, identifying 204 wavelength data may include calculating the normalized levels of wavelengths of light sensed for each data set collected. FIG. 13 schematically depicts illustrative plots 92 of the three hundred data sets of normalized levels of the wavelengths of the full spectrum of light collected (e.g., from about 400 nm to about 700 nm), where wavelength in nanometers is on the X-axis and the normalized levels of wavelengths (e.g., the reflectance in percentage) are on the Y-axis. Once the normalized levels of the wavelengths of light are determined for each data set, an average normalized level of the wavelengths of light in each data set is calculated (e.g., to identify three hundred averages, where there are 300 hundred data sets), as discussed further herein. Other suitable techniques may be utilized to identify wavelength data, as desired.

The method 200, as depicted in FIG. 12, may include associating or matching 206 portions of the identified wavelength data to one or more reactants of the reactant array 26 on the surface of the CSA 24. The associating or matching portions of the identified wavelength data to one or more reactants may be achieved in any suitable manner utilizing determined data from the fluid analysis system 10 and/or user input (e.g., a user may input when a reactant is being scanned by the light collection component 16, which reactant is being scanned by the light collection component 16, etc.). In some examples and similar to as discussed above, the determined wavelength data may be associated with one or more reactants of the reactant array 26 based on, for example, a combination of a known time of when a test started, a known rate at which the CSA is adjusted, and when the light was collected from the CSA 24, running a data analysis (e.g., one or more algorithms) on the wavelength data, and/or by associating or matching the wavelength data with one or more reactants in one or more other suitable manners.

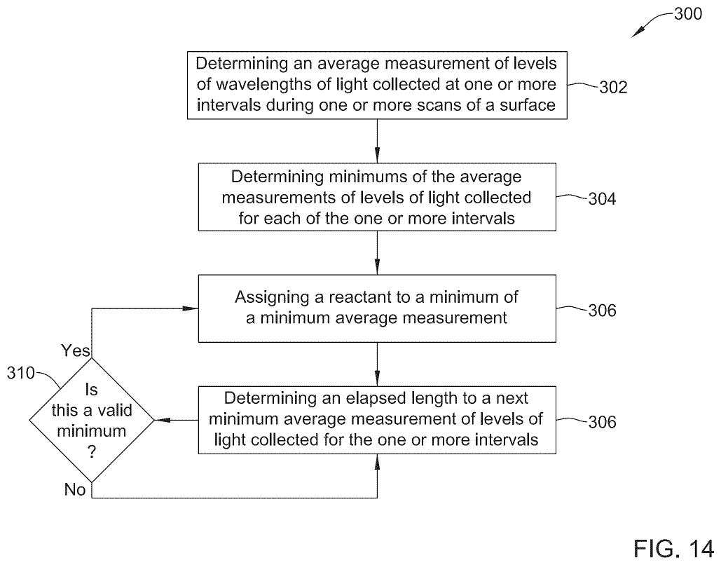

FIG. 14 depicts a schematic diagram of an illustrative technique 300 for matching or associating wavelength data (e.g., levels of the wavelengths of light that are determined over time and/or other suitable sets of data determined based on the levels of the wavelengths of light) with one or more reactants as part of a fluid analysis test. Although the technique 300 for matching or associating wavelength data with one or more reactants may be utilized in one or more other scenarios, we will discuss the steps of the technique 300 relative to the above example discussed with steps 202 and 204 of the method 200, where three hundred wavelength data sets were identified with each data set including normalized levels of wavelengths for a full spectrum of light.

The technique 300 may include determining 302, during or after a fluid analysis test, an average measurement of the levels of wavelengths of light collected (e.g., light from across the full spectrum of light collected) at one or more intervals during one or more scans of the surface of the CSA 24. In the example of the fluid analysis test discussed above in which three hundred wavelength data sets were identified, the average normalized levels of wavelengths of light for each of the three hundred data sets may be determined. Optionally, once the average measurement of the levels of (e.g., normalized levels of) the wavelengths of light for each data set have been determined, these values may be saved as part of a respective data set on which the average measurement value is based and/or saved separate from the respective data set.

Once the averages of the levels of wavelengths of light collected have been determined, each minimum value of the averages from the data sets may be determined 304. In some examples, minimum average values of the data sets may be identified when the averages values of the data sets are organized sequentially by identifying the average values that are less than average values immediately before and after the average value. However, other suitable algorithms may be identified (e.g., second derivative functions). The minimum average values of the levels of the wavelengths of light collected may be determined to be associated with a reactant of a reactant array, as the reactants of the CSA 24 may be separated by a lighter colored surface (e.g., separated by a white colored surface), which will have data sets with higher average levels of wavelengths of light collected than data sets collected from a reactant that is non-white.

To facilitate identifying the minimum average values, the average values for all of the data sets of levels of wavelengths of light collected may be plotted on a graph. In the example discussed, all three hundred average values from the three hundred data sets may be plotted and the minimum values may be identified from the plot by highlighting and/or otherwise noting data sets or sample number and values of the levels of wavelengths of light collected that are associated with valleys of the plot.

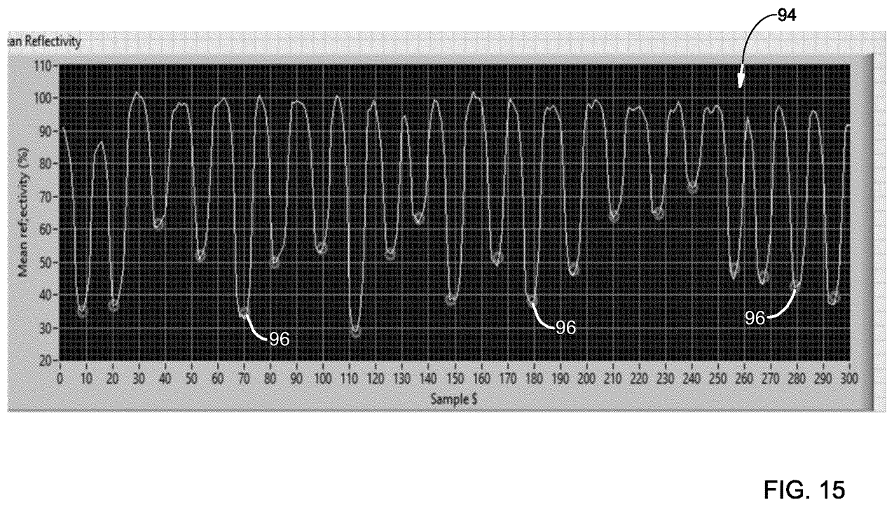

FIG. 15 schematically depicts a plot 94 of the three hundred average values ordered sequentially with respect to when during the fluid analysis test the light associated therewith was collected, with the minimum average values and data set numbers associated with the minimum average values highlighted with circles 96 (only some circles are labeled with 96 for clarity purposes). In FIG. 15, the sample number or data set number is on the X-axis and the average normalized levels of wavelengths of light collected are on the Y-axis. In some examples, rather than list a sample number or data set number on the X-axis, the X-axis may provide times when light associated with the data sets was collected, cumulative distances the substrate had traveled when light associated with the data sets was collected, and/or other suitable sequential values related to when light associated with the data sets was collected. Software and/or user observations may be utilized to identify the minimum average values from the data sets.

Along with the measurements of average values of levels of the wavelengths of light collected, an indication of the minimum average values of levels of wavelengths may be stored with or relative to associated data sets. Further, additional information may be identified and/or stored with or relative to the respective data sets in the controller 18 or at one or more location(s) in communication with the controller 18 that includes, but is not limited to, a time at which a data set was obtained (e.g., an elapsed time since a start of the fluid analysis test), a speed or rate at which the CSA 24 is adjusted relative to the light collection component 16, a length (in distance, mm) of a single scan during the fluid analysis test, a length (in distance, mm) of all of the scans during the fluid analysis test, a spacing (in distance, mm) between adjacent reactants, data set numbers and reactant numbers associated with a first minimum average value of the data sets for each of the first two scans and for the last scan, a data set number for a last valid minimum average value of all of the data sets, a tolerance value for a maximum variation of the spacing between adjacent reactants of the reactant array 26, and/or other suitable data associated with the reactants of the CSA 24, a sizing of the CSA 24, data sets collected or determined, and/or an operation of the fluid analysis system 10. The information stored at or in communication with the controller 18 may be obtained automatically from the system 10 and/or in response to user input.

Data set numbers may be assigned sequentially starting with n and adding one for each sequential data set obtained throughout the fluid analysis test. Reactant numbers may be assigned sequentially along the CSA 24 in any suitable manner.

The technique 300, as depicted in FIG. 14, may include assigning 306 a first identified minimum average value of levels of wavelengths of light collected to a first reactant on the CSA 24. Then, determine 308 an elapsed length (e.g., in time or distance) until or to a next minimum average value of levels of wavelength of light collected and determine at a validation step 310 if the elapsed length indicates the next identified minimum average value is associated with a reactant on the CSA 24. A valid minimum average value may be identified when the length, in distance, is within a maximum tolerance of a distance between two adjacent reactants, when the length, in time, is within an expected elapsed time between data collected from adjacent reactants, and/or based on other information related to the CSA 24 and/or the operation of the fluid analysis system 10. In some examples, determining an elapsed length to a next minimum average value may include determining a time at which each minimum average value occurred and/or determining a location at which each minimum average value occurred (e.g., an elapsed time or distance of each minimum average value since the fluid analysis test was initiated).

If the validation step 310 indicates the minimum average value is not a valid minimum associated with a reactant, the technique 300 may return to determining 308 an elapsed length to a next minimum average value and proceed with the technique 300, as discussed. In some examples, the minimum average value that is not a valid minimum may be discarded or ignored. If the validation step 310 indicates the minimum average value is a valid minimum associated with a reactant, the technique 300 may return to assigning 306 the valid average minimum value and/or an associated data set to the next reactant and proceed with the technique 300. The technique 300 may procced in a loop until all reactants of each scan have been associated with a minimum average value and/or an associated data set.

Although photons originating from diffuse reflections of light directed at the CSA from the illumination component 12 haven been primarily referred to herein, the fluid analysis system 10 may be adapted for use in measuring the spectrum of emitted photons resulting from the conversion to lower energy photons by the absorption and re-emission process of florescent decay. There are many possible variations of the implementation of a florescent spectral measurement device. The excitation (often called pump) illumination can be a constant wave or pulsed and wideband or narrowband at selected wavelengths tailored to the dyes employed for a specific color bar and the specific compound or gas under analyses. In addition to detecting the specific wavelength or wavelengths of the fluorescently emitted discharge photons, a time based waveshape of the photon pulse emitted from a short duration high peak power excitation source such as a gas flashlamp, electron discharge x-ray source, or pulsed ultraviolet or blue shifted visible laser may be measured utilizing a fast photo-detector with or without a Fabry-Perot or other type of wavelength filter. The phase shift or time-based spectrum change of an amplitude or otherwise modulated constant wave light source can also be detected. Variations of these concepts are contemplated.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Unless otherwise expressly stated, it is in no way intended that any method or technique set forth herein is to be construed as requiring that its steps be performed in a specific order. This holds for any possible non-express basis for interpretation, including matters of logic with respect to arrangement of steps or operational flow, plain meaning derived from grammatical organization or punctuation, and the number or type of embodiments described in the specification.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system comprising:
a light source directed at a surface;
a spectrometer configured to measure, over time, levels of wavelengths of light collected from the surface; and
a controller in communication with the spectrometer, and
wherein the controller is configured to identify a component of fluid in contact with the surface based on the levels of the wavelengths of light collected from the surface.

2. The system of claim 1, wherein the light source comprises a broadband white light emitting diode (LED).

3. The system of claim 1, wherein the light source comprises a lens having a diameter and a focal length, wherein a ratio of the diameter to the focal length is one.

4. The system of claim 1, wherein the light source comprises light having a wavelength spanning a range of 400 nanometers (nm) to 725 nm.

5. The system of claim 1, further comprising:
one or more optical fibers in communication with the spectrometer and configured to collect light from the light from the surface and direct the light collected to the spectrometer.

6. The system of claim 1, further comprising:
a light collection component configured to collect the light from the surface; and
an adjustable stage configured to be moved relative to the light collection component, and
wherein the adjustable stage is configured to support a component having the surface.

7. The system of claim 6, further comprising:
a motor in communication with the adjustable stage and configured to move the adjustable stage relative to the light collection component.

8. The system of claim 1, wherein the controller being configured to identify the component of fluid in contact with the surface based on the levels of the wavelengths of light collected from the surface comprises the controller being configured to identify the component of fluid in contact with the surface based on one or more both of a timing of the levels of the wavelengths of light reflected off of the surface and an absolute change between a level of a wavelength of light collected from the surface at a time prior to an application of the fluid to the surface and at a predetermined time after initially applying the fluid to the surface.

9. A method comprising:
adjusting a substrate along a light collection component in communication with a controller, wherein the substrate supports one or more reactants configured to change color in response to exposure to one or more fluids of interest;
exposing the one or more reactants to a fluid;
determining, over time with the controller, levels of wavelengths of light collected by the light collection component; and
determining a component of the fluid based on the levels of the wavelengths of light collected.

10. The method of claim 9, further comprising:
applying light to the substrate, wherein the light comprises light from a broadband white light emitting diode.

11. The method of claim 10, wherein applying light to the substrate comprises applying light from a first angle and a first location with respect to the substrate and a second angle and a second location with respect to the substrate, where the second angle is the same as the first angle and the second location is different than the first location.

12. The method of claim 9, wherein adjusting the substrate along the light collection component comprises passing the substrate along the light collection component a plurality of passes, wherein each pass of the plurality of passes comprises passing a predetermined number of the one or more reactants on the substrate along the light collection component.

13. The method of claim 9, further comprising:
associating the levels of the wavelengths of light collected by the light collection component with the one or more reactants.

14. The method of claim 13, wherein determining a component of the fluid based on the levels of the wavelengths of light collected comprises determining the component of the fluid based on one or both of a timing of the levels of the wavelengths of light collected as associated with the one or more reactants and an absolute change between levels of a wavelength of light collected as associated with the one or more reactants prior to the beginning of an application of the fluid to the one or more reactants and at a predetermined time after initially applying the fluid to the one or more reactants.

15. The method of claim 9, wherein determining, over time with the controller, the levels of the wavelengths of light collected by the light collection component comprises determining levels of a spectra of the light collected spanning wavelengths in a range of 425 nm to 725 nm.

16. The method of claim 9, wherein determining the component of the fluid based on the levels of the wavelengths of light collected comprises:
determining statistical data for levels of the wavelengths of light collected at a plurality of instances of time; and
comparing the statistical data determined to predetermined component statistical data.

17. A non-transitory computer readable medium having stored thereon a program code for use by a computing device, the program code causing the computing device to execute a method for determining a component of a fluid, the method comprising:
determining levels of wavelengths of light collected by a light collection component at one or more intervals, wherein the light collected during at least one interval is at least partially from one or more reactants;
associating one or more of the levels of the wavelengths of light collected with one of the one or more reactants;
comparing the levels of the wavelengths associated with the one or more reactants to predetermined sets of levels of the wavelengths for the one or more reactants, wherein each predetermined set is associated with a component of a fluid; and
when the levels of the wavelengths associated with the one or more reactants matches a predetermined set of levels of the wavelengths for the one or more reactants, identifying the component of the fluid associated with the predetermined set of levels of the wavelengths for the one or more reactants.

18. The non-transitory computer readable medium of claim 17, wherein associating one or more of the levels of the wavelengths of light collected with one of the one or more reactants comprises:
determining a time at which a minimum of the levels of the wavelengths of light collected occurred; and
associating a reactant of the one or more reactants with the levels of the wavelengths of light collected at the time.

19. The non-transitory computer readable medium of claim 18, wherein the determining the time at which the minimum of the levels of the wavelengths of light collected occurred and the associating a reactant of the one or more reactants with the levels of the wavelengths of light collected at the time are repeated until all levels of the wavelengths of light collected at times associated with a minimum of levels are associated with a reactant of the one or more reactants or are discarded as being invalid minimums.

20. The non-transitory computer readable medium of claim 18, wherein the minimum of the levels of the wavelengths of light collected is a minimum of an average level of light over an entire spectrum of light collected.
